(19) United States
(12) Patent Application Publication
(10) Pub. No.: US 2024/0050617 A1
(43) Pub. Date: Feb. 15, 2024

(54) FRAGRANCE DELIVERY DEVICES, AND METHODS OF MAKING AND USING THE SAME

(71) Applicant: NEBUSCENT, LLC, Watkinsville, GA (US)

(72) Inventors: Michael T. Foley, Watkinsville, GA (US); Randall Marc Bachtel, Lawrenceville, GA (US); James Richard Cargile, Smyrna, GA (US); Taylor Kopacka Leigh, Milton, GA (US); Jason Lye, Atlanta, GA (US); Stephen Lawrence Phelps, Lilburn, GA (US); Peter Wyndham Shipp, Woodstock, GA (US); Michael Allan Sloan, Atlanta, GA (US); Noah Dawson Underwood, Atlanta, GA (US)

(21) Appl. No.: 18/233,532

(22) Filed: Aug. 14, 2023

Related U.S. Application Data
(60) Provisional application No. 63/450,761, filed on Mar. 8, 2023.
(60) Provisional application No. 63/397,536, filed on Aug. 12, 2022.

(51) Int. Cl. A61L 9/12

(52) U.S. Cl. CPC A61L 9/12; A61L 2209/133; A61L 2209/16; A61L 2209/134

(57) ABSTRACT

Fragrance delivery devices are disclosed. The fragrance delivery devices are designed to couple with an existing air supply of a room, such as a patient care room, and utilize air flow from the existing air supply to disperse a fragrance-carrying fluid from the fragrance delivery device into a surrounding environment.

Methods of making and using fragrance delivery devices are also disclosed.

### CROSS-REFERENCE TO RELATED APPLICATIONS

This patent application claims the benefit of priority to (i) U.S. Provisional Patent Application Ser. No. 63/450,761 filed on Mar. 8, 2023 entitled “FRAGRANCE DELIVERY DEVICES, AND METHODS OF MAKING AND USING THE SAME,” and (ii) U.S. Provisional Patent Application Ser. No. 63/397,536 filed on Aug. 12, 2022 entitled “FRAGRANCE DELIVERY DEVICES, AND METHODS OF MAKING AND USING THE SAME,” the subject matter of both of which is hereby incorporated by reference in its entirety.

### FIELD OF THE INVENTION

The present invention relates to air fragrance delivery devices and methods of using the same. In particular, devices and methods according to the present invention address the delivery of air fragrance in medical settings and other settings, using a source of compressed gas, such as a compressed gas cylinder or pre-installed compressed gas sources within the medical setting and other setting.

### BACKGROUND OF THE INVENTION

Unpleasant odors can cause discomfort and unease, and may create an immediate and long-lasting impression for those who experience such odors. If an individual experiences an unpleasant odor in connection with a business entity, then that individual may develop an overall negative impression of that entity, regardless of the quality of the goods or services provided, and may forego engaging with that entity or may not return for subsequent transactions.

The stigma of unpleasant odors can be especially problematic for healthcare facilities, which are visited daily by individuals of varying hygienic habits, for the delivery of a wide array of medical procedures. Differences in personal hygiene of the many visiting individuals, as well as distinct odors associated with some medical supplies, may result in healthcare facilities having strong odors that some may find unpleasant. The presence of such unpleasant odors may cause individuals to avoid visiting healthcare facilities, potentially forgoing medical treatment. Unpleasant odors at a healthcare facility may also interfere with the delivery of medical care, for example, by causing such discomfort and unease to a patient that they experience changes to their breathing and/or heart rate, as well as potentially distracting medical staff from focusing on the patient and/or the treatment.

Accordingly, there is a need for fragrance delivery devices that mask or remove unpleasant odors, such as in healthcare facilities, so that patients may be at ease during their visit and to avoid undo distractions to medical staff that serve the patients.

### SUMMARY OF THE INVENTION

The present invention is directed to fragrance delivery devices. The fragrance delivery devices of the present invention utilize a source of compressed gas, such as a compressed gas cylinder or an existing air supply line of a room, such as in a patient care room, to disperse a fragrance-carrying fluid into an atmosphere outside the fragrance delivery device, and into the room. As used herein, the term “fragrance” is used to describe compositions that (i) have their own distinct smell/odor, and/or (ii) mask or hide one or more smells or odors and/or (iii) neutralize, sequester, bind, absorb, adsorb, complex, oxidize, or otherwise chemically alter an odor to render it odorless.

The fragrance delivery devices of the present invention comprise: a body defining an interior space, the body comprising a nozzle coupler designed to be attachable to a source of compressed gas, such as an existing air supply line of a room (for example, in a patient care room) or a separate compressed gas supply (for example, a compressed gas cylinder), the fragrance delivery device being adapted to utilize pneumatic pressure from the air or compressed gas supply to disperse a fragrance-carrying fluid into an atmosphere outside the fragrance delivery device.

In some embodiments, the fragrance delivery device of the present invention comprises a fragrance delivery device comprising: a body defining an interior space, the body comprising a nozzle coupler designed to be attachable to a source of compressed gas, such as an existing air supply line of a patient care room (or other room) or a compressed gas cylinder, the fragrance delivery device being adapted to utilize pneumatic pressure from a source of compressed gas, such as the existing air supply line to disperse a fragrance-carrying fluid into an atmosphere outside the fragrance delivery device. The pneumatic pressure from a source of compressed gas can be used to force a fragrance-carrying from the fragrance delivery device into an atmosphere outside the fragrance delivery device. Alternatively, the pneumatic pressure from a source of compressed gas can be intermixed with a fragrance medium to form and then disperse a fragrance-carrying fluid from the fragrance delivery device.

In some desired embodiments, the fragrance delivery device of the present invention comprises a fragrance delivery device comprising: a body defining an interior space, the body comprising a nozzle coupler designed to be attachable to a source of compressed gas, such as a compressed gas cylinder or an existing air supply line of a patient care room, the fragrance delivery device being adapted to (1) utilize pneumatic pressure from a source of compressed gas, such as a compressed gas cylinder or the existing air supply line to generate an intensified (e.g., an increased) pressure within a disposable cartridge containing a liquid, and (2) utilize the intensified pressure to disperse a fragrance-carrying fluid into an atmosphere outside the fragrance delivery device.

In some desired embodiments, any of the herein-described fragrance delivery devices of the present invention disperses and/or dispenses a fragrance-carrying fluid as a fine mist into an atmosphere outside the fragrance delivery device without using any internal or external electric power to disperse and/or dispense the fragrance-carrying fluid, namely, the control of whether the device is on (i.e., dispersing and/or dispensing) or off (i.e., not dispersing and/or dispensing) does not require any electronic components, and is accomplished by mechanical components as detailed below; e.g., the body is only connected to a source of compressed gas, such as a compressed gas cylinder or the existing air supply line of a patient care room or other room.

In some embodiments, the fragrance delivery device of the present invention comprises a fragrance delivery device comprising: a body defining an interior space, the body comprising (i) a nozzle coupler designed to be attachable to a source of compressed gas, such as a compressed gas cylinder or an existing air supply line of a patient care room (or other room), and (ii) a removable cartridge port sized to accept a removable cartridge containing a fragrance medium, the nozzle coupler being adapted to receive a first gas flow G1 from a source of compressed gas, such as a compressed gas cylinder or the existing air supply line and utilize the first gas flow G1 to disperse a fragrance-carrying fluid from a removable cartridge positioned within the removable cartridge port, the removable cartridge comprises one or more cartridge nozzles sized to allow passage of the fragrance-carrying fluid from the removable cartridge into an atmosphere outside the fragrance delivery device. In some desired embodiments, the fragrance delivery device comprises one or more dispensers that generate and deliver an increased hydraulic pressure inside the removable cartridge so as to force the fragrance-carrying fluid from the one or more cartridge nozzles of the removable cartridge into an atmosphere outside the fragrance delivery device through one or more nozzles.

The fragrance dispensing devices of the present invention may be utilized with a compressed gas source, with the fragrance dispensing device placed in fluid communication with the gas source via a gas supply unit such as those conventionally found at healthcare facilities and/or other facilities (i.e., any location with an existing airline and a need to eliminate odors). The fragrance dispensing device may receive a compressed gas flow G1 from the gas source, via the gas supply unit, through a mating connection between the nozzle coupler of the fragrance dispensing device and an outlet nozzle of the gas supply unit. A separate connector may also be used to connect the nozzle coupler of the fragrance dispensing device and an outlet nozzle of the gas supply unit (e.g., plastic tubing).

As discussed herein, the fragrance dispensing device of the present invention may be powered using medical compressed air supplied to most beds in hospitals, or compressed gas cylinders, or an independent air compressor. The device may optionally include one or more gas pressure regulators, optional additional on-off valving, and optional pressure indicator gauges.

The present invention is further directed to methods of making fragrance dispensing devices. In some embodiments, the method of making a fragrance dispensing device comprises: forming a fragrance delivery device comprising: a body defining an interior space, the body comprising a nozzle coupler designed to be attachable to a source of compressed gas, such as a compressed gas cylinder or an existing air supply line of a patient care room, wherein the fragrance delivery device is designed to (1) receive a first gas flow G1 from a source of compressed gas, and (2) utilize a pneumatic pressure of the first gas flow G1 to disperse and/or dispense a fragrance-carrying fluid into an atmosphere outside the fragrance delivery device.

In some embodiments, the method of making a fragrance dispensing device comprises: forming a fragrance delivery device comprising: a body defining an interior space, the body comprising a nozzle coupler designed to be attachable to a source of compressed gas, such as a compressed gas cylinder or an existing air supply line of a patient care room, the fragrance delivery device being adapted to utilize a pneumatic pressure from a source of compressed gas, such as a compressed gas cylinder or the existing air supply line to disperse (and/or dispense) a fragrance-carrying fluid into an atmosphere outside the fragrance delivery device, preferably, without using any internal or external electric power to disperse the fragrance-carrying fluid into a fine mist, namely, the control of whether the device is on (i.e., dispersing and/or dispensing) or off (i.e., not dispersing and/or dispensing) does not require any electronic components, and is accomplished by mechanical components as detailed below; e.g., the body is only connected to a source of pressurized gas, such as a compressed gas cylinder or the existing air supply line of a patient care room or other room, or a gas cylinder.

In some embodiments, the method of making a fragrance dispensing device comprises: forming a fragrance delivery device comprising: a body defining an interior space, the body comprising (i) a nozzle coupler designed to be attachable to a source of compressed gas, such as a compressed gas cylinder or an existing air supply line of a patient care room (or other room), and (ii) a removable cartridge port sized to accept a removable cartridge containing a fragrance medium, the nozzle coupler being adapted to receive a first gas flow G1 from a source of compressed gas, such as a compressed gas cylinder or the existing air supply line or from another source of compressed gas and utilize a pneumatic pressure of the first gas flow G1 to disperse (and/or dispense) a fragrance-carrying fluid from one or more nozzles within the removable cartridge and sized to allow passage of the fragrance-carrying fluid from the removable cartridge to an atmosphere outside the fragrance delivery device. Desirably, the fragrance delivery device is formed so as to disperses the fragrance-carrying fluid without using any internal or external electric power to disperse the fragrance-carrying fluid, namely, the control of whether the device is on (i.e., dispersing and/or dispensing) or off (i.e., not dispersing and/or dispensing) does not require any electronic components, and is accomplished by mechanical components as detailed below; e.g., the body is only connected to a source of compressed gas, such as a compressed gas cylinder or the existing air supply line of a patient care room or other room, or to an alternate compressed gas source.

The present invention is even further directed to methods of using fragrance dispensing devices. In some embodiments, the method of using a fragrance dispensing device comprises: connecting a fragrance delivery device to a source of compressed gas, such as a compressed gas cylinder or an existing air supply line of a patient care room or to an alternate compressed gas source, the fragrance delivery device comprising: a body defining an interior space, the body comprising a nozzle coupler designed to be attachable to a source of compressed gas, such as a compressed gas cylinder or the existing air supply line of a patient care room or another compressed gas source, wherein the fragrance delivery device (1) receives a first gas flow G1 from a source of compressed gas, such as a compressed gas cylinder or the existing air supply line or other source, and (2) utilizes a pneumatic pressure of the first gas flow G1 to disperse and/or dispense a fragrance-carrying fluid into an atmosphere outside the fragrance delivery device.

In some embodiments, the method of using a fragrance dispensing device comprises: connecting a fragrance delivery device to a source of compressed gas, such as a compressed gas cylinder or an existing air supply line of a patient care room (or other room) or to an alternate compressed gas source, the fragrance delivery device comprising: a body defining an interior space, the body comprising (i) a nozzle coupler designed to be attachable to a source of compressed gas, such as for example an existing air supply line of a patient care room (or other room) or a compressed gas cylinder, and (ii) a removable cartridge port sized to accept a removable cartridge containing a fragrance medium, the nozzle coupler being adapted to receive a first gas flow G1 from a source of compressed gas, such as a compressed gas cylinder or the existing air supply line or to an alternate compressed gas source and utilize a pneumatic pressure of the first gas flow G1 to disperse and/or dispense a fragrance-carrying fluid from one or more nozzles within the removable cartridge and sized to allow passage of the fragrance-carrying fluid from the removable cartridge to an atmosphere outside the fragrance delivery device. Desirably, the method disperses the fragrance-carrying fluid into a fine mist without using any internal or external electric power to disperse the fragrance-carrying fluid, namely, the control of whether the device is on (i.e., dispersing and/or dispensing) or off (i.e., not dispersing and/or dispensing) does not require any electronic components, and is accomplished by mechanical components as detailed below; e.g., the body is only connected to a source of compressed gas, such as the existing air supply line of a patient care room or other room, or a compressed gas cylinder.

The foregoing general description and the following detailed description are exemplary and explanatory only and are intended to provide further explanation of the invention as claimed. The accompanying drawings are included to provide a further understanding of the invention; are incorporated in and constitute part of this specification; illustrate embodiments of the invention; and, together with the description, serve to explain the principles of the invention.

### BRIEF DESCRIPTION OF THE DRAWINGS

Further features and advantages of the invention can be ascertained from the following detailed description that is provided in connection with the drawings described below:

FIG. 1 illustrates a conventional gas supply unit that may be used with fragrance devices according to the present invention;

FIG. 2 illustrates a fragrance device according to the present invention;

FIG. 3 illustrates another fragrance device according to the present invention;

FIG. 4 illustrates another fragrance device according to the present invention;

FIG. 4A illustrates cartridge loading, locking, and unloading features of the exemplary fragrance device shown in FIG. 4;

FIG. 4B illustrates activation of a housing lever to rewind a spring to restart the timing mechanism of the exemplary fragrance device shown in FIG. 4;

FIG. 5 illustrates another fragrance device according to the present invention;

FIG. 6 illustrates cartridge dispensing features of the fragrance device shown in FIG. 5;

FIG. 7 illustrates a cartridge loading feature of the fragrance device shown in FIG. 5;

FIG. 8 illustrates an assembly of mechanical components according to the present invention, the assembly of mechanical components being suitable for use in the fragrance devices of the present invention such as the fragrance devices shown in FIGS. 4-7;

FIG. 9 illustrates a rear view of the assembly of mechanical components shown in FIG. 8;

FIG. 10 illustrates a close-up view of some of the mechanical components shown in FIG. 9;

FIG. 11 illustrates a left side view of the assembly of mechanical components shown in FIG. 8;

FIG. 12 illustrates a close-up, right side view of some of the fragrance device components within the assembly of mechanical components shown in FIG. 8;

FIG. 13 illustrates a cross-sectional view of an exemplary pressure intensifier suitable for use in the fragrance devices shown in FIGS. 4-7, such as the exemplary pressure intensifiers shown in FIGS. 8 and 11-12;

FIGS. 13A-13C illustrate exploded views of exemplary pistons suitable for use within the exemplary pressure intensifier shown in FIG. 13 with (i) FIG. 13A showing an exemplary piston moving towards the exemplary removable cartridge shown in FIG. 13, (ii) FIG. 13B showing the exemplary piston moving away from the exemplary removable cartridge shown in FIG. 13, and (iii) FIG. 13C showing a cross-sectional view of another exemplary piston suitable for use within a pressure intensifier shown in FIG. 13, and providing detail of an o-ring/o-ring groove system of the exemplary piston;

FIGS. 14A-14B illustrate detailed views of a removable cartridge suitable for use in the fragrance devices of the present invention with FIG. 14B providing a cut-away top perspective view of the exemplary removable cartridge 130′ shown in FIG. 14A;

FIG. 14C provides an alternative cut-away top perspective view of another exemplary removable cartridge 130′ shown in FIG. 14A;

FIG. 15 illustrates an air flow layout of the exemplary assembly of mechanical components shown in FIG. 8;

FIG. 15A illustrates a frontal view of an exemplary rotating cam shown in FIG. 15;

FIG. 16 illustrates rotating cams suitable for use in the exemplary timing mechanism shown in FIG. 9;

FIG. 17 illustrates an exemplary device connector suitable for use between (i) a conventional gas supply unit, such as the exemplary conventional gas supply unit shown in FIG. 1, and (ii) a fragrance device according to the present invention;

FIG. 18 illustrates a fragrance device according to the present invention in combination with a compressed gas container containing a compressed gas;

FIG. 19 illustrates a cross-sectional view of an exemplary cam shaft that could be an alternative to the cam shaft shown in FIGS. 9-10;

FIG. 20 illustrates an alternate embodiment in which the rack-n-pinion assembly shown in FIGS. 9-10 is replaced with a gear attached to a lever;

FIGS. 21A-21B depict two different perspective views of some of the fragrance device components within an assembly of mechanical components used to form an alternate fragrance device according to the present invention; and

FIG. 22 depicts a cut-away view of an exemplary fragrance device according to the present invention with a fragrance delivery cartridge installed therein.

### DETAILED DESCRIPTION OF THE INVENTION

The following disclosure discusses the present invention with reference to the examples shown in the accompanying drawings, though does not limit the invention to those examples.

The use of any and all examples, or exemplary language (e.g., “such as”) provided herein is intended merely to better illuminate the present invention and does not pose a limitation on the scope of the present invention unless otherwise claimed. No language in the specification should be construed as indicating any non-claimed element as essential or otherwise critical to the practice of the present invention unless made clear in context.

As used herein, the singular forms “a,” “an,” and “the” include plural referents unless the context clearly dictates otherwise. Unless indicated otherwise by context, the term “or” is to be understood as an inclusive “or.” Terms such as “first,” “second,” “third,” etc. when used to describe multiple devices or elements, are so used only to convey the relative actions, positioning and/or functions of the separate devices, and do not necessitate either a specific order for such devices or elements, or any specific quantity or ranking of such devices or elements.

Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and/or with one or more disclosed steps omitted.

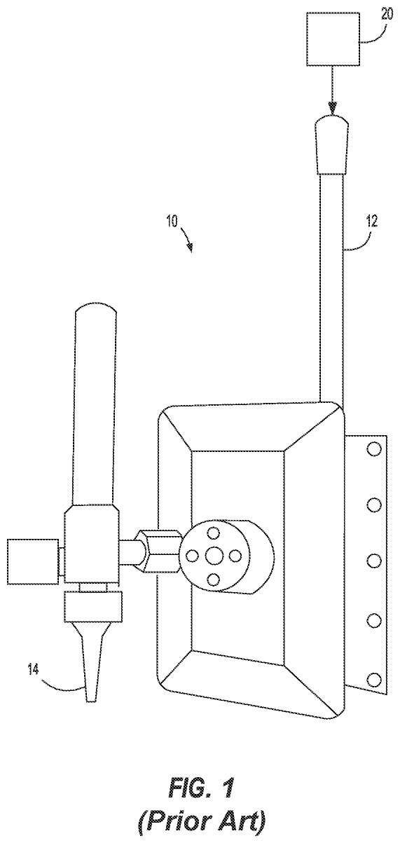

FIG. 1 shows one example of a compressed gas supply unit 10 that is commonly available at many healthcare facilities. The compressed gas supply unit 10 includes a gas line 12 that feeds a compressed gas from a gas source 20 that stores a compressed gas, and a nozzle 14 for ejecting the compressed gas from the gas source 20 via the gas line 12.

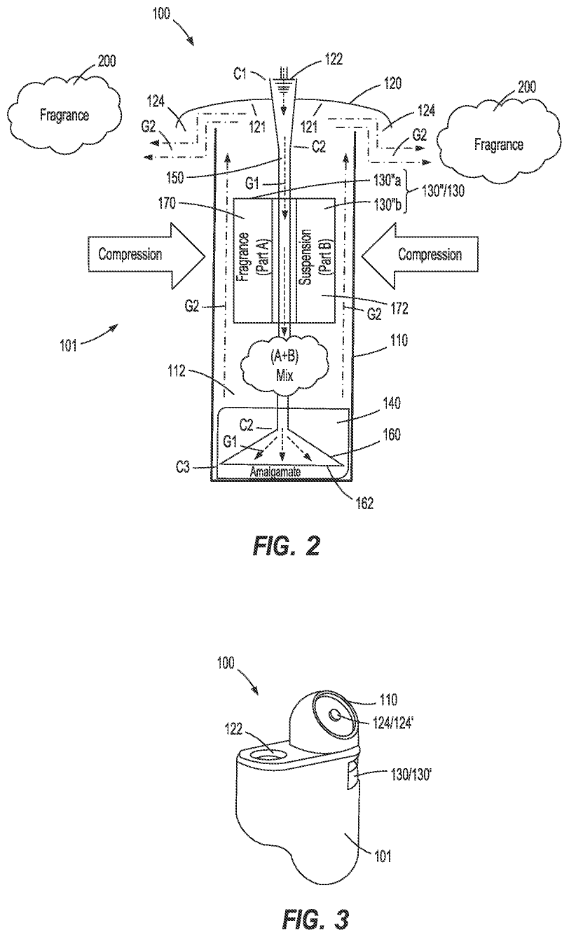

FIG. 2 shows an example of one type of fragrance delivery device 100 according to the present invention. As shown in FIG. 2, device 100 has a body 101 that includes an outer casing 110 and an upper canopy 120. The outer casing 110 and canopy 120 may both be formed in a cylindrical shape, as shown, though may also be made in any number of other shapes. The outer casing 110 and canopy 120 may be formed as two separate structures that are releasably connectable to one another, or may be formed as a single monolithic structure.

Provided within the outer casing 110 is an interior space 112 that houses a plurality of frangible containers 130″. As shown in FIG. 2, there may be provided a first frangible container 130″a containing a “Part A” medium that acts as a fragrance component, and a second frangible container 130″b containing a “Part B” medium that acts as a suspension component. Preferably, both mediums are liquids, though any suitable medium (and combinations of mediums) may be used, including though not limited to, the use of gases and powders.

The device 100 is adapted to facilitate fracturing of the frangible containers 130″, so as to allow mixing of the separate Part A and Part B components to form a fragrance-carrying amalgamate 140 at a bottom region of the interior space 112. The device 100 may be so adapted by constructing the outer casing 110 of a flexible material such that a user may apply a compression force to the casing 110, for example by manually squeezing, so as to crush the frangible containers 130″ within the interior space 112. In such a construction, the outer casing 110 is preferably made with a flexible material having sufficient elasticity such that, upon cessation of the compression force, the casing 110 rebounds to the same shape it had in the unstressed state, prior to application of the compression force. Alternatively, the device 100 may be constructed with pincer mechanisms or spring-loaded pins that fracture the frangible containers 130″ upon activation of a switch or dial on an external surface of the casing 110.

The canopy 120 is joined to an upper end of the outer casing 110 and includes a nozzle coupler 122 for coupling with a nozzle 14 of a gas supply unit 10, such as that shown in FIG. 1. The nozzle coupler 122 is in fluid communication with a stem 150 that extends downward through the interior space 112 of the outer casing 110, passing centrally between the frangible containers 130″, and terminating at a diffuser 160 positioned at a bottom region of the interior space 112. The diffuser 160 is adapted to disperse and emit a gas flow G1 received from the stem 150 into a lower region of the interior space 112. This may be achieved by constructing the diffuser 160 with a generally conical shape having a perforated base 162, as illustrated in FIG. 2; though the diffuser 160 may be formed in any other suitable shapes, including though not limited to spherical or oval shapes with perforations provided over a portion or an entirety thereof.

Preferably, the nozzle coupler 122 is formed as a funnel that reduces from a first larger cross-section C1 at an opening thereof to a second smaller cross-section C2 at an outlet thereof where the coupler 122 connects to the stem 150. The stem 150 then maintains the reduced second cross-section C2 along a length thereof, with the diffuser 160 being formed as an inverted funnel that expands from the reduced second cross-section C2 at an opening that connects to the stem 150 to a third enlarged cross-section C3 at an outlet of the diffuser 160. With such a construction, the nozzle coupler 122, stem 150 and diffuser 160 provide a Venturi structure that further promotes dispersal of a compressed gas flow G1 into the lower region of the interior space 112.

As shown in FIG. 2, the canopy 120 may extend around an entire periphery of the upper end of the outer casing 110, with one or more vents 124 positioned between the casing 110 and the canopy 120. The one or more vents 124 may be formed as a single structure that extends around an entire periphery of the device 100 in the form of a screen that is suitably adapted to both support the canopy 120 on the casing 110 and provide an egress therethrough for a fragrance-carrying gas flow G2 to exit from the interior space 112 to an atmosphere 200 outside the device 100. Alternatively, the canopy 120 may be supported on the casing 110 by multiple struts (not shown) that are spaced equidistantly around the periphery of the device 100, with open spaces between the struts serving as vents 124 for the passage of the gas flow G2. An interior surface 121 of the canopy 120, facing toward the interior space 112 of the casing 110, is provided with a sloped surface that arcs toward the vent(s) 124 so as to provide a surface that directs and guides a rising gas flow G2 to the vent(s) 124.

The one or more vents 124 are not limited to those positions illustrated in FIG. 2, and vents (not shown) may also be provided, in addition to or in place of those as illustrated, at one or more locations along the outer casing 110 and/or the canopy 120. In such examples, the interior space 112 may also include one or more internal structures (e.g., sloped surfaces)(not shown) for directing the rising gas flow G2 to these vents.

In use, the device 100 will be placed in fluid communication with a gas source 20 by joining the nozzle coupler 122 to a nozzle 14 of a gas supply unit 10. Thereafter a user may manipulate the outer casing 110 to fracture the frangible containers 130″ within the interior space 112, either by applying a compression force to the casing 110 or by activating another frangible container fracturing mechanism. Upon fracturing the containers 130″, the Part A and Part B components will then mix to form a fragrance-carrying amalgamate 140 that will reside at a bottom region of the interior space 112, submerging at least a perforated base portion 162 of the diffuser 160. The user may then activate the gas supply unit 10 to feed a gas flow G1 to the nozzle coupler 122, through the stem 150, and out the diffuser 160. Dispersion of the compressed gas flow G1 through the perforated portion 162 of the diffuser 160, while submerged in the amalgamate 140, will agitate and vaporize the amalgamate 140 thereby forming a fragrance-carrying gas flow G2. The fragrance-carrying gas flow G2 will then rise within the interior space 112, along interior walls of the casing 110, until it reaches the canopy 120, where the gas G2 will then be guided by the interior sloped surface 121 to the one of more vents 124 for egress to an atmosphere 200 outside the device 100, thereby emitting a fragrance to the surrounding environment.

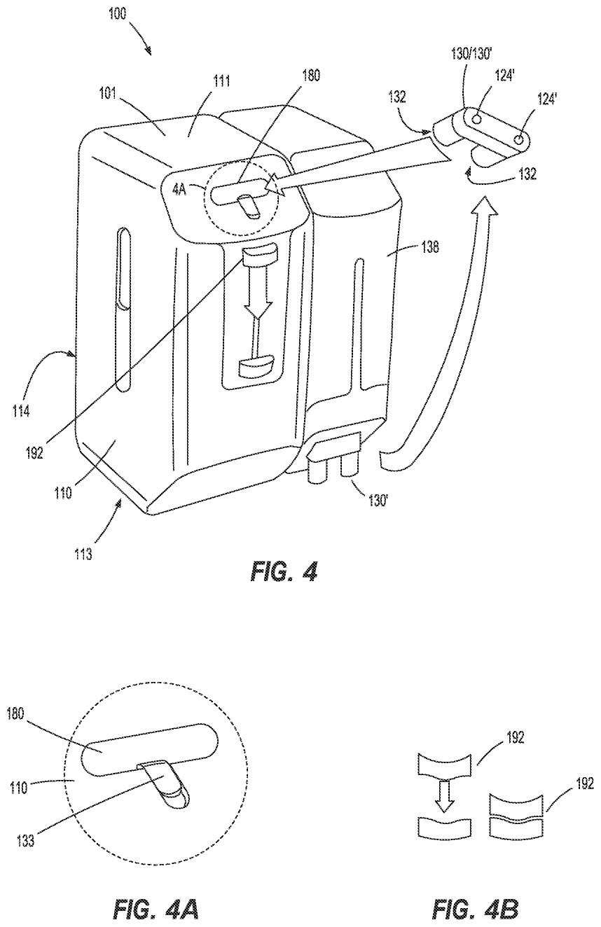

FIG. 3 shows an outer housing configuration of an exemplary fragrance delivery device 100 according to the present invention. The fragrance delivery device 100 has a body 101 that includes an outer casing 110, a nozzle coupler 122, and one of more vents 124. In some embodiments, one or more frangible containers 130″ may be positioned within an interior space 112 (not shown) of fragrance delivery device 100. In other embodiments, one or more removable cartridge 130′ may be positioned within a removable cartridge port 180 (not shown in FIG. 3, but discussed below) along an outer surface of the outer casing 110 of the fragrance delivery device 100.

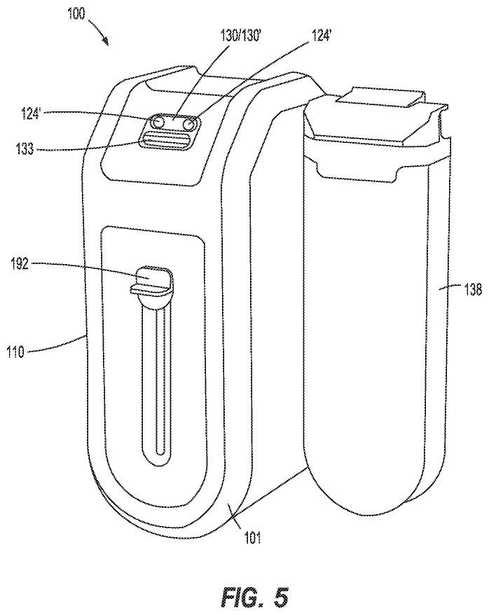

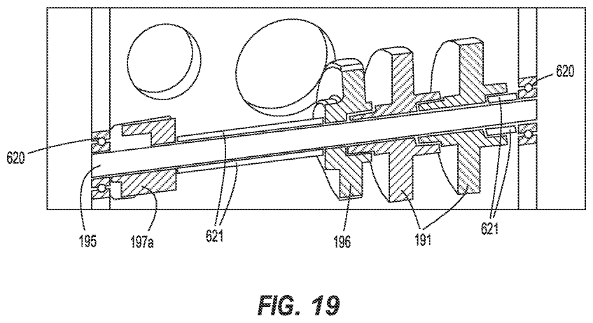

FIG. 4 shows yet another more preferred type of fragrance delivery device 100 according to the present invention. This fragrance delivery device 100 has a body 101 that includes an outer casing 110. Although a nozzle coupler 122 is not shown in FIG. 4, it should be understood that a nozzle coupler 122 could be positioned along any outer surface of outer casing 110 (e.g., casing top surface 111, casing bottom surface 113, or casing rear surface 114). As shown in FIG. 4, the exemplary fragrance delivery device 100 comprises a removable cartridge port 180 sized to accept a removable cartridge 130′ containing a fragrance medium 170. The nozzle coupler 122 of the fragrance delivery device 100 is designed to receive a first gas flow G1 from a source of compressed gas, such as a compressed gas cylinder 600 (see, FIG. 18) or an existing air supply line 12 (see, FIG. 1) and utilize a pneumatic pressure of the first gas flow G1 to disperse (and/or dispense) a fragrance-carrying fluid from the removable cartridge 130′. As shown, the removable cartridge 130′ comprises one or more nozzles 124′ sized to allow passage of the fragrance-carrying fluid from the removable cartridge 130′ into an atmosphere 200 outside the fragrance delivery device 100.

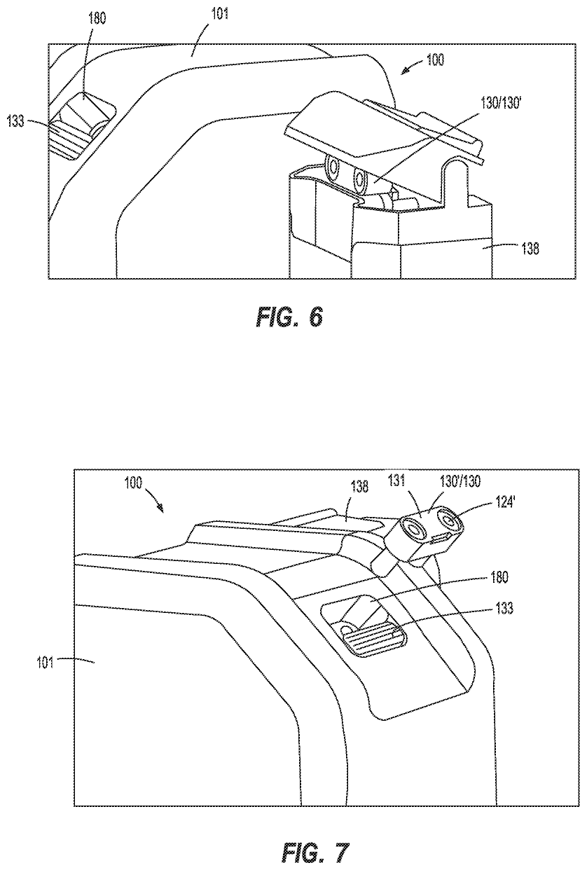

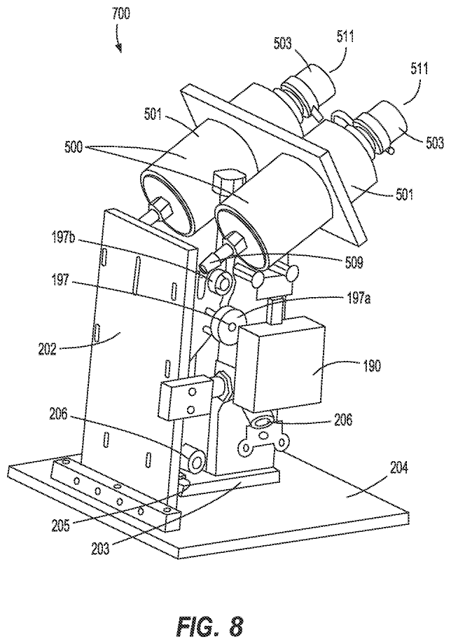

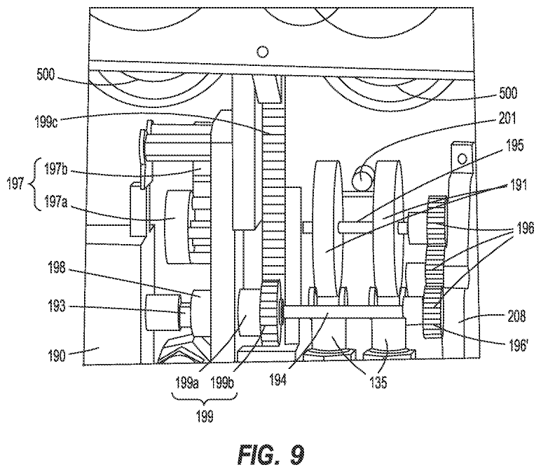

FIGS. 5-7 show other examples of fragrance delivery devices 100 according to the present invention. Like the exemplary fragrance delivery device 100 shown in FIG. 4, the exemplary fragrance delivery device 100 shown in FIGS. 5-7 comprises a removable cartridge port 180 sized to accept a removable cartridge 130′ containing a fragrance medium 170. Although a nozzle coupler 122 is not shown in FIGS. 5-7, it should be understood that a nozzle coupler 122 could be positioned along any outer surface of outer casing 110 (e.g., casing top surface 111, casing bottom surface 113, or casing rear surface 114), and the fragrance delivery device 100 be designed to receive a first gas flow G1 from a source of compressed gas, such a compressed gas cylinder 600 or an existing air supply line 12 and utilize a pneumatic pressure of the first gas flow G1 to disperse a fragrance-carrying fluid from the removable cartridge 130′ through one or more nozzles 124′.

The fragrance delivery devices 100 of the present invention may be operated in a number of different ways. In some embodiments, the fragrance delivery device 100 may be used to emit a constant flow of fragrance-carrying fluid over an extended period of time by applying the pneumatic pressure of the incoming gas flow G1 for the extended period of time (e.g., up to about 30 minutes). The fragrance delivery device 100 may also be used to emit one or more quick, bursts of fragrance-carrying fluid, with each burst lasting only a few seconds, i.e., a reduced period of time (e.g., from about one to three second), over a period of time by controlling the pneumatic pressure of the incoming gas flow G1 for the period of time (e.g., activate and quickly deactivate one or more valves 35). The fragrance delivery device 100 may be used to emit fragrance-carrying fluid into the air directly over the patient, or adjacent to the patient, or to some other area of the room. The fragrance delivery device 100 may be used to emit fragrance-carrying fluid into the air in a designated part of the room, pre-determined by the presence of ventilation ducts, exhaust fan inlets, and or heating and air conditioning ducts. Ideally, air flow in the room may be measured by installers beforehand, so that the fragrance delivery device 100 can be located in the position that disperses the fragrance carrying fluids most efficiently based upon the existing airflow within the room.

Though some embodiments of the present invention are described with a construction of the fragrance delivery device 100 that provides (1) a single pair of frangible containers 130″, for example, with only Part A and Part B components, for use in a single activation, or (2) a single removable cartridge 130′, it should be understood that the fragrance delivery devices 100 of the present invention are not limited to such constructions. The fragrance delivery devices 100 may be adapted to use any number of frangible containers 130″ and/or removable cartridges 130′ in a single activation—such as (a) constructions in which there are three frangible containers 130″, with the third container providing a third component (e.g., a Part C), such as a sanitizing substance, for inclusion in the emitted fragrance-carrying fluid, or (b) constructions in which a given removable cartridge 130′ comprises one to more separate fragrance-carrying fluid reservoirs 412 positioned within the removable cartridge 130′. The fragrance delivery devices 100 may also be adapted to contain multiple groupings of frangible containers 130″ (e.g., multiple pairs of containers 130″, or multiple collections of three or more containers 130″) or multiple collections of two or more removable cartridge 130′, with individual container groupings available for separate activation from one another, thereby enabling multiple activations of a single fragrance delivery device 100.

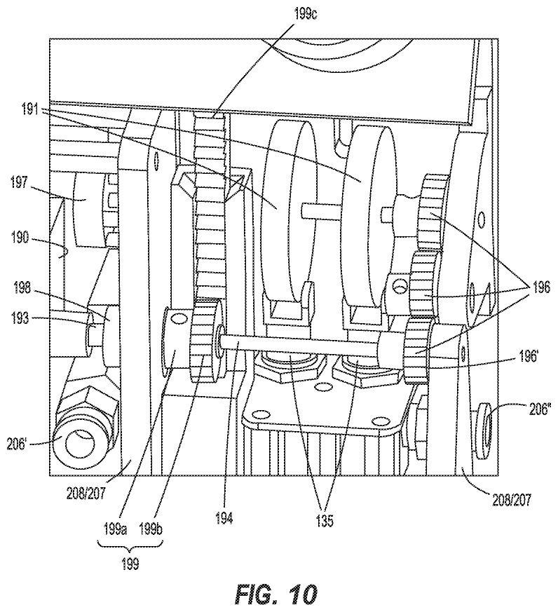

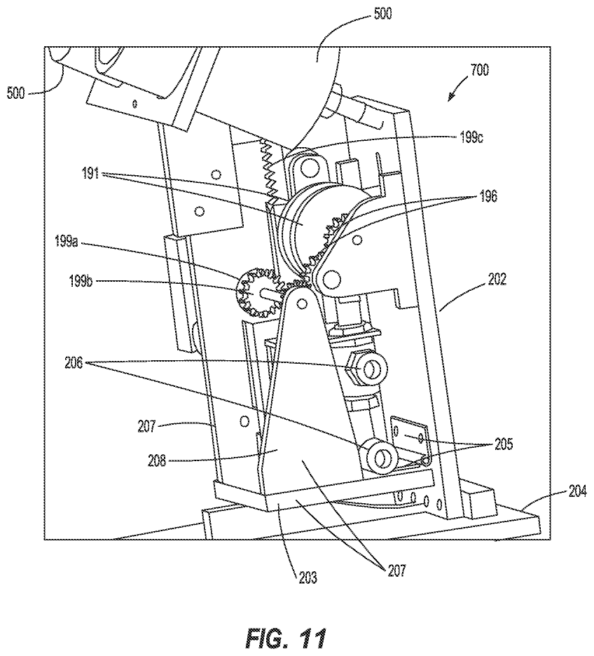

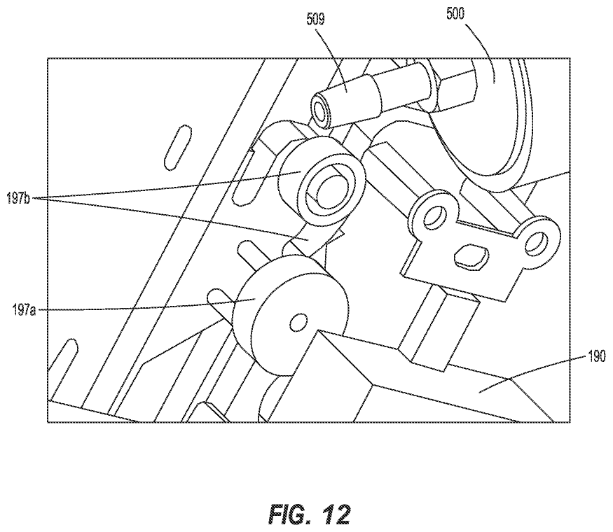

FIG. 8 provides a side perspective view of an exemplary assembly of mechanical components 700 suitable for use in the fragrance delivery devices 100 according to the present invention. FIG. 9 provides a front view of the exemplary assembly of mechanical components 700 shown in FIG. 8. FIG. 10 provides a close-up view of some of the fragrance device components shown in FIG. 9.

As shown in FIGS. 8-10, exemplary assembly of mechanical components 700 comprises: (a) a timing mechanism 190 that runs for a desired length of time (e.g., from 2.0 seconds to 60 minutes, typically about a 30 minute duration, while providing approximately 210 degrees of output shaft 193 rotation thru this 30 minute cycle), (b) an output shaft 193 extending from the timing mechanism 190, and rotating for a desired length of time (e.g., from 2.0 seconds to 60 minutes)(see, FIGS. 9-10), (c) a driver shaft 194 that is axially connected to the output shaft 193 (see, FIGS. 9-10), (d) a cam shaft 195, which is in an “offset” position relative to the driver shaft 194 (see, FIGS. 9-10), (e) a combination of spur gears 196 that connect the driver shaft 194 to the cam shaft 195 (e.g., the cam shaft 195 is “disconnected” from the driver shaft 194 when the combination of spur gears 196 are separated, which allows a coil spring assembly 197 to rotate the cam shaft 195 back to a “reset/start” position; when the driver shaft 194 is “connected” to the cam shaft 195, the cam shaft 195 also rotates for a desired length of time (e.g., from 2.0 seconds to 60 seconds))(see, FIGS. 9-10), (f) one or more cam gears 191 designed to (i) rotate when the cam shaft 195 rotates, and (i) open and close one or more valves 135 during rotation (see, FIGS. 9-10), (g) the one or more valves 135 that control when pneumatic pressure is applied to disperse a fragrance-carrying fluid 270 (not shown in FIG. 8)(see, FIGS. 9-10) into an atmosphere 200 outside the fragrance delivery device 100, (h) a coil spring assembly 197 designed to rotate the cam shaft 195 back to a “reset/start” position following a dispersion step, the coil spring assembly 197 comprising (h)(i) a cam shaft reset wheel 197a, and (h)(ii) a coiled spring 197b, (i) an driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198 (see, FIGS. 9-10), ( ) a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows the pinon gear 199b to spin in an opposite unwind direction (see, FIGS. 9-10), (k) an adjustable stop pin 201 that provides a correct rotation start position for the cam shaft 195 (see, FIG. 9), and (l) one or more pressure intensifiers 500, each pressure intensifier 500 being capable of generating an intensified (e.g., an increased) hydraulic pressure used to disperse fragrance-carrying fluid 270 into an atmosphere 200 outside the fragrance delivery device 100.

Although not illustrated in FIGS. 9-10, it should be understood that one or more additional assist springs may be added to assist with the rotation of cam shaft 195 and increase torque. The one or more optional assist springs may be included so as to act upon the drive shaft 194 to assist in rotation, which then acts through the separable spur gears 196 to assist in the rotation of the cam shaft 195. In addition, it may prove advantageous to move the spur gears 196 connecting the drive shaft 194 to the cam shaft 195 from one end of the drive shaft 194 to the other so that the gear train is more compact. In order to increase timing choices, gears 196 with more teeth may be used between the cam shaft 195 and the drive shaft 194. Further, to assist the meshing of gears 196 when the cam shaft 195 and the drive shaft 194 are moved into contact, the teeth may be formed with rounded tips instead of flat.

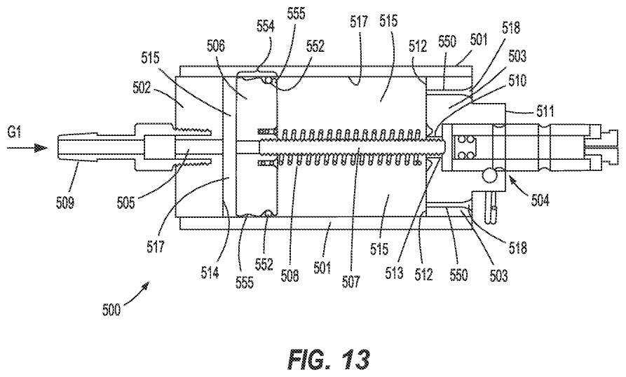

All of the above-mentioned components within exemplary assembly of mechanical components 700 are either (1) connected to (directly or indirectly) a fixed (i.e., stationary) vertical base plate 202, attached to a fixed (i.e., stationary) horizontal base plate 204, or (2) positioned on and connected to a pivoting plate 203, the pivoting plate 203 being connected, via a hinge 205, to the fixed vertical base plate 202 (see, FIGS. 8 and 11). The position of the pivoting plate 203 can be controlled with a lever or button (e.g., spring-rewinding lever 192) positioned along an outer surface of an outer casing 110 of the fragrance delivery device 100. In some embodiments, the position of the pivoting plate 203 may be controlled by the insertion of a removable cartridge 130′ containing the fragrance into the fragrance delivery device 100. The pivoting plate 203 may be forced from one position to another by the insertion of a wedge between pivoting plate 203 and fixed (i.e., stationary) horizontal base plate 204. For example, in some embodiments, when the pivoting plate 203 is horizontal, the exemplary assembly of mechanical components 700 is “active” and (1) the main air pressure from a source of compressed gas, such a compressed gas cylinder 600 or existing air supply 14 is ON (e.g., air supply valve 134 from gas supply 12 is open), and (2) the combination of spur gears 196 that connect the driver shaft 194 to the cam shaft 195 are engaged (i.e., connected with one another). In these embodiments, when the pivoting plate 203 pivots downward, two things happen simultaneously: (1) the main air pressure from a source of compressed gas, such a compressed gas cylinder 600 or an existing air supply 14 is OFF (e.g., air supply valve 134 from gas supply 12 is closed), and (2) the combination of spur gears 196 that connect the driver shaft 194 to the cam shaft 195 are disengaged (i.e., not connected with one another).

FIG. 11 provides a lefthand view of the exemplary assembly of mechanical components 700 shown in FIG. 8. FIG. 11 provides a better view of hinge 205 connecting the pivoting plate 203 to the fixed vertical base plate 202. FIG. 11 also provides a better view of a pivoting plate assembly 207 that comprises: pivoting plate 203, and a vertically-extending pivot plate 208 connected to pivoting plate 203 so as to move with pivoting plate 203. Vertically-extending pivot plate 208 providing support for one or more of: (a) timing mechanism 190, (b) output shaft 193, (c) driver shaft 194, (d) at least one spur gear 196′ within the combination of spur gears 196, (e) the one or more valves 135, (f) the coil spring assembly 197, (g) the driver shaft clutch 198, and (h) the rack-n-pinion rewind assembly 199. In addition, FIG. 11 provides a better view of air line connections 206, which provide an air inlet (i.e., supply) and an air outlet (i.e., exhaust) for the one or more valves 135 (e.g., two air line connections 206 per side as shown in FIGS. 8, 10, and 11). It should be noted that although air tubing is used with the exemplary assembly of mechanical components 700 shown in FIGS. 8-11, air tubing is not shown in these figures.

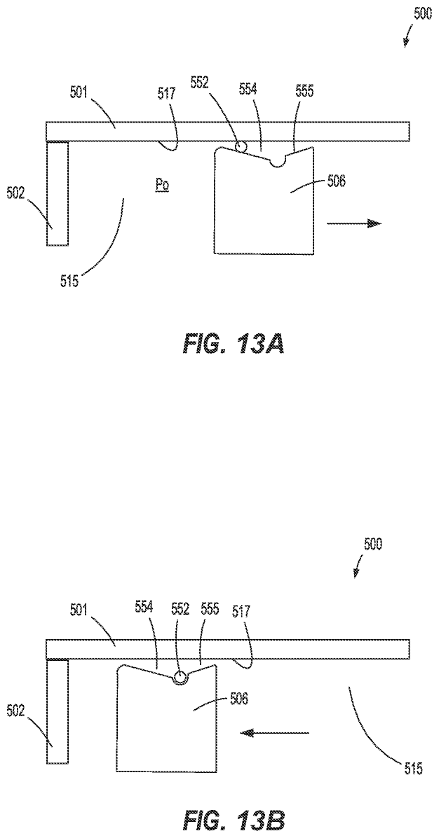

FIG. 12 provided a closer view of the coil spring assembly 197 shown in FIG. 9. As shown in FIG. 12, coil spring assembly 197 comprising (i) a cam shaft reset wheel 197a (also referred to as spoked cam shaft wheel 197a) and (ii) a coiled spring 197b. Cam shaft reset wheel 197a (also referred to as spoked cam shaft wheel 197a) and coiled spring 197b work together to rewind/“reset” the cam shaft 195 to a “start position” when the combination of spur gears 196 that connect the driver shaft 194 to the cam shaft 195 are “dis-engaged”. As further shown in FIG. 12, pressure intensifier 500 has a pneumatic pressure intensifier air inlet coupling member 509 that enables an airline connector (e.g., plastic tubing, not shown) to be connected to the pressure intensifier 500.

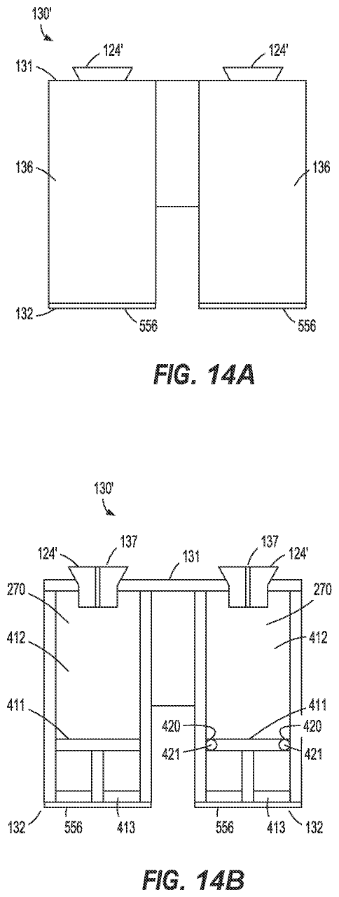

FIG. 13 provided a cross-sectional view of an exemplary pressure intensifier 500 in combination with an exemplary removable cartridge 130′. As shown in FIG. 13, exemplary pressure intensifier 500 comprises the following components: (1) a master cylinder side wall 501, (2) a bottom end cap 502, (3) a top end cap 503, (4) a top end cap slot 504 within an outer surface 511 of the top end cap 503, the top end cap slot 504 being sized to accept at least a portion of an impact surface end 132 of a given removable cartridge 130′, (5) a pressure intensifier air inlet coupling member 509, (6) a bottom end cap air channel 505, (7) a pneumatic pressure intensifier piston 506, (8) a connecting rod 507 attached to the pneumatic pressure intensifier piston 506, (9) a return spring 508 positioned on and encircling at least a portion of the connecting rod 507, (10) a top end cap channel 510 within an inner surface 512 of the top end cap 503, top end cap channel 510 being sized to receive a connecting rod impact end 513 of the connecting rod 507, and (11) a master cylinder volume 515 bound by an inner surface 517 of the master cylinder side wall 501, a bottom end cap inner surface 514 of the bottom end cap 502, and the top end cap 503, and the inner surface 512 of the top end cap 503.

During a dispersion step, first gas flow G1 from a source of compressed gas, such as a compressed gas cylinder 600, or an existing air supply line 12 enters pressure intensifier air inlet coupling member 509, and applies pneumatic pressure P_{O} onto pressure intensifier piston 506, which pushes the connecting rod 507 so as to push on a cartridge piston 411 of a removable cartridge 130′ so as to force fragrance-carrying fluid 270 from one or more cartridge nozzles 124′ of the removable cartridge 130′ and into an atmosphere 200 outside the fragrance delivery device 100. An intensified (e.g., an increased) pressure P_{I} may be obtained, the intensified (e.g., an increased) pressure P_{I} being greater than the pneumatic pressure P_{O} of the first gas flow G1 from a source of compressed gas, such as a compressed gas cylinder 600, or the existing air supply line 12, by adjusting overall dimensions of the pressure intensifier piston 506, and the cartridge piston 411 of a removable cartridge 130′. Given that:

P_{O}×A_{PO}=P_{I}×A_{PC},

where A_{PO} represents a piston surface area of pressure intensifier piston 506, and A_{PC} represents a piston surface area of the cartridge piston 411 of a removable cartridge 130′, pressure P_{I} can be increased by either (1) increasing the piston surface area of pressure intensifier piston 506, A_{PO}, or (2) decreasing the piston surface area of the cartridge piston 411 of the removable cartridge 130′, A_{PC}. In some embodiments of the present invention, intensified (e.g., an increased) pressure P_{I} is from about 2 to 40 times greater than the pneumatic pressure P_{O} of the first gas flow G1 from a source of compressed gas, such as the existing air supply line 12. In other embodiments, of the present invention, intensified (e.g., an increased) pressure P_{I} is from about 10 to 30 times greater than the pneumatic pressure P_{O} of the first gas flow G1 from a source of compressed gas, such as the existing air supply line 12. More preferably, the ratio of P₁ to P₀ is in the range of 16:1 to 25:1. Most preferably, the ratio of P₁ to P₀ is 24:1. At a P₁ to P₀ ratio of 24:1, with hospital air supplied at 55 psi, the maximum pressure P₁ possible in the fluid filled cavity 412 at the time of dispensing/dispersing is 1,320 psi, neglecting losses due to friction.

During a dispersing event, it may be important for air to escape from master cylinder volume 515 and this is permitted by utilizing numerous air vents 550 that allow air to flow out of master cylinder volume 515 as the pressure intensifier piston 506 advances as shown in FIG. 13A, and to refill the master cylinder volume 515 with air as the pressure intensifier piston 506 resets due to the spring 508 when the air G1 is released as shown in FIG. 13B. Air vents 550 may be straight, or they may be conical have a larger diameter on inner surface 512 of the top end cap 503 than on the exterior surface 518 of the top end cap 503, or they may be inverted to have a smaller diameter on inner surface 512 versus on the exterior surface 518. In some embodiments, it may be advantageous to reduce the size of the air vents 550 thereby restricting the escaping air into a jet to further disperse the fragrance-carrying fluid 270 into the room (i.e., atmosphere 200).

FIGS. 13A-13B illustrate exploded views of exemplary pressure intensifier piston 506 moving within exemplary pressure intensifier 500. FIG. 13A shows exemplary pressure intensifier piston 506 moving towards exemplary removable cartridge 130′, while FIG. 13B shows exemplary pressure intensifier piston 506 moving away from exemplary removable cartridge 130′.

FIGS. 13A and 13B also provide detail views showing the use of o-ring 552 to further seal exemplary pressure intensifier piston 506 against inner surface 517 of the master cylinder side wall 501. It should be understood that one or more o-rings 552 may lay within one or more o-ring grooves 554 that circumscribe the outer side surface 555 of exemplary pressure intensifier piston 506. Typically, two o-rings 552 or more provide a better seal, and more piston stability during operation.

FIG. 13B shows exemplary pressure intensifier piston 506 resetting in position following a dispersal event. As shown in FIG. 13B, exemplary pressure intensifier piston 506 is moving under the force of spring 508 once air pressure (i.e., the pneumatic pressure P_{O} of the first gas flow G1) is released. Due to the shaping of o-ring groove 554, when exemplary pressure intensifier piston 506 is moving toward the bottom end cap 502 (i.e., away from exemplary removable cartridge 130′), the o-ring 552 may be less pressed against inner surface 517 of the master cylinder side wall 501, because no seal is necessary when resetting, and reduced frictional force allows for the use of a lighter return spring 508. As shown in FIG. 13A, the o-ring groves 554 may be shaped in such a way that the o-ring 552 is pressed more firmly against inner surface 517 of the master cylinder side wall 501 when exemplary pressure intensifier piston 506 is advancing toward exemplary removable cartridge 130′. This feature improves the seal of exemplary pressure intensifier piston 506 under pressure.

FIG. 13C shows an exemplary pressure intensifier piston 506 in position prior to a dispersal event. As shown in FIG. 13C, exemplary pressure intensifier piston 506 moves under the force of spring 508 once air pressure (i.e., the pneumatic pressure P_{O} of the first gas flow G1) is released. Due to the shape of o-ring grooves 554, when exemplary pressure intensifier piston 506 is moving toward the bottom end cap 502 (i.e., away from exemplary removable cartridge 130′), the one or more o-rings 552 are less pressed against inner surface 517 of the master cylinder side wall 501, because no seal is necessary when resetting, and reduced frictional force allows for the use of a lighter return spring 508. Reduced frictional force also reduces wear and tear on the one or more o-rings 552 due to repeated motions. As shown in FIG. 13A, the one or more o-ring groves 554 may be shaped in such a way that the one or more o-rings 552 is pressed more firmly against inner surface 517 of the master cylinder side wall 501 when exemplary pressure intensifier piston 506 is advancing toward exemplary removable cartridge 130′. This feature improves the seal of exemplary pressure intensifier piston 506 under pressure.

This piston-sealing feature including the movement of the one or more o-rings 552 into a position of stronger seal when traveling in one direction versus another may have application and usefulness beyond the pressure intensifiers 500 used in the present invention. A similar configuration could potentially be used in a hand pneumatic air pump (not shown), in which case the air pressure on the downstroke requires a greater seal than the upstroke in which the pump cylinder is filled with fresh air. Similarly, the piston-sealing feature in which o-ring 552 movement is used to cause a difference in seal pressure when the piston is moving in one direction versus another could be applied to other inventions, such as liquid pumps (not shown). For instance, a hand operated pump on a well (not shown) may include such a mechanism in which, on the upstroke, when a person operating the pump pushes down on the handle, the piston begins to move up the inside of the pump chamber. This forces the o-ring 552 against the pump chamber wall (not shown) with additional force, maintaining a vacuum needed to draw water from depth. In a fourth example, automobile air conditioner compressors work by pushing rods in and out of cylinders to create pressure and pump coolant through condenser and evaporator coils. This same technology (i.e., piston-sealing feature) could be applied to such automotive coolant compressors with potential efficiency and wear savings.

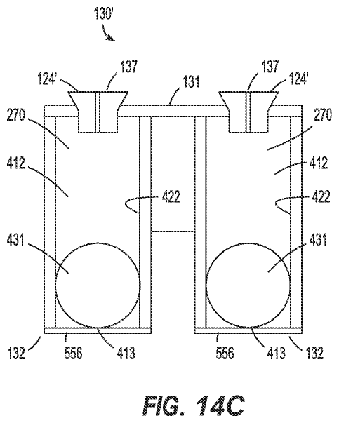

FIGS. 14A-14C provide more details of an exemplary removable cartridge 130′ suitable for use in the fragrance devices 100 of the present invention. FIG. 14A provides a top perspective view of exemplary removable cartridge 130′. As shown in FIG. 14A, exemplary removable cartridge 130′ comprises a cartridge body 136 having an outer surface end 131, and an impact surface end 132 opposite the outer surface end 131. The removable cartridge 130′ further comprises one or more cartridge nozzles 124′ extending from the outer surface end 131, and sized to allow passage of a fragrance-carrying fluid 270 (contained within one or more cavities 412) from the removable cartridge 130′ into an atmosphere 200 outside the fragrance delivery device 100.

FIG. 14B provides a cut-away top perspective view of the exemplary removable cartridge 130′ shown in FIG. 14A. As shown in FIG. 14B, exemplary removable cartridge 130′ further comprises: (1) two separate cartridge pistons 411, one for each fragrance-carrying fluid reservoir 412 positioned within the removable cartridge 130′, (2) two fragrance-carrying fluid reservoirs 412, (3) two separate nozzle outlets 137, one for each nozzle 124′, and (4) two separate cartridge impact surface members 413, one for each fragrance-carrying fluid reservoir 412 and each cartridge piston 411. Optionally, the cartridge pistons 411 may include one or more groves 420 that seat one or more o-rings 421 that assist in sealing the piston against the cartridge inner surface 422 during release of the fragrance carrying fluid. Optionally, a frangible membrane 556 may be placed over impact surface end 132. When connecting rod impact end 513 of the connecting rod 507 is driven forward through top end cap channel 510 by air pressure P₀ acting on exemplary pressure intensifier piston 506, connecting rod impact end 513 pierces the optional frangible membrane 556 and drives cartridge piston 411 along fragrance-carrying fluid reservoir 412, dispensing/dispersing the fragrance-carrying fluid 270 contained therein. As discussed above, it should be understood that a given removable cartridge 130′ may comprise a single fragrance-carrying fluid reservoir 412 positioned within the removable cartridge 130′, or may comprise two or more fragrance-carrying fluid reservoirs 412 positioned within the removable cartridge 130′.

FIG. 14C provides a cut-away top perspective view of another exemplary removable cartridge 130′ shown in FIG. 14A. As shown in FIG. 14C, exemplary removable cartridge 130′ further comprises: (1) two separate cartridge pistons 431 that, in this embodiment, take the form of tight fitting rubber spherical balls 431, one for each fragrance-carrying fluid reservoir 412 positioned within the removable cartridge 130′, (2) two fragrance-carrying fluid reservoirs 412, (3) two separate nozzle outlets 137, one for each nozzle 124′, and (4) two separate cartridge impact surface members 413, one for each fragrance-carrying fluid reservoir 412 and each cartridge piston 431. The use of a ball 431 (instead of using an essentially cylindrical piston 411 with o-rings 421) simplifies assembly because no o-rings 421 are needed, and simplifies assembly because a ball 431 does not need to be oriented in a specific direction prior to insertion. The ball 431 may optionally be lubricated to assist in travel along the inner surface 422.

Optionally, a frangible membrane 556 may be placed over impact surface end 132. When connecting rod impact end 513 of the connecting rod 507 is driven forward through top end cap channel 510 by air pressure P₀ acting on exemplary pressure intensifier piston 506, connecting rod impact end 513 pierces the optional frangible membrane 556 and drives cartridge piston/ball 431 along fragrance-carrying fluid reservoir 412, dispensing/dispersing the fragrance-carrying fluid 270 contained therein. As discussed above, it should be understood that a given removable cartridge 130′ may comprise a single fragrance-carrying fluid reservoir 412 positioned within the removable cartridge 130′, or may comprise two or more fragrance-carrying fluid reservoirs 412 positioned within the removable cartridge 130′.

During a dispersion step, a pneumatic pressure P_{O} from first gas flow G1 from a source of compressed gas, such as a compressed gas cylinder 600, or the existing air supply line 12 or an intensified (e.g., an increased) pressure P_{I} pushes on cartridge impact surface member 413 or directly on cartridge connecting rod 414, which forces cartridge piston/ball 411/431 onto a fragrance-carrying fluid 270 within a fragrance-carrying fluid reservoir 412, which forces fragrance-carrying fluid 270 out of one or more cartridge nozzles 124′. Cartridge spring 508 i.e., return spring 508) provides enough spring force to reset the exemplary pressure intensifier piston 506 when the air pressure P_{O} is released.

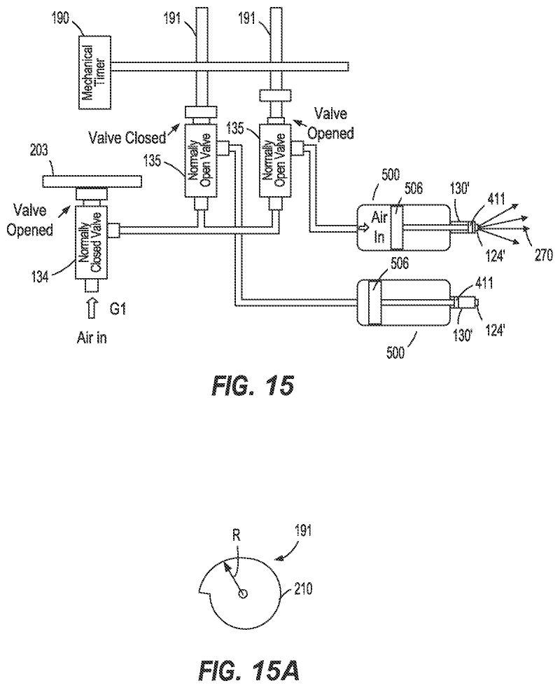

FIG. 15 provides an exemplary air flow layout of the exemplary assembly of mechanical components 700 shown in FIG. 8. As shown in FIG. 15; a first gas flow G1 from a source of compressed gas, such as a compressed gas tank 600, or an existing air supply line 12 (not shown) passes through an air supply valve 134 when air supply valve 134 is in an “open” position, for example, when the pivoting plate 203 of the exemplary assembly of mechanical components 700 is in a horizontal position, as described above, and the combination of spur gears 196 that connect the driver shaft 194 to the cam shaft 195 are engaged (i.e., connected with one another). First gas flow G1 then applies a pneumatic pressure P_{O} onto one or more valves 135. The one or more valves 135 open or close based on a rotation position of one or more cam gears 191. Rotation of the one or more cam gears 191 is controlled by timing mechanism 190, which, when activated, rotates the one or more cam gears 191 for a desired length of time, for example, from as little as 5.0 seconds to 60 minutes or more. As shown in FIG. 15, the one or more valves 135 are “closed” when a portion of the cam gear 191 having a greater radius is in contact with the one or more valves 135 (i.e., the valve 135 on the left-hand side of FIG. 15), and are “open” when a portion of the cam gear 191 having a smaller radius is in contact with the one or more valves 135 (i.e., the valve 135 on the right-hand side of FIG. 15).

FIG. 15A provides a frontal view of exemplary cam gear 191 shown in FIG. 15. As shown in 15A, cam gear 191 has a radius R that varies along the periphery 210 of cam gear 191. While FIG. 15A shows a particular shape for exemplary cam gear 191, it should be understood that a given cam gear 191 may have any desired peripheral shape so as to result in one or more times that the one or more valves 135 are opened during a given rotation of the given cam gear 191.

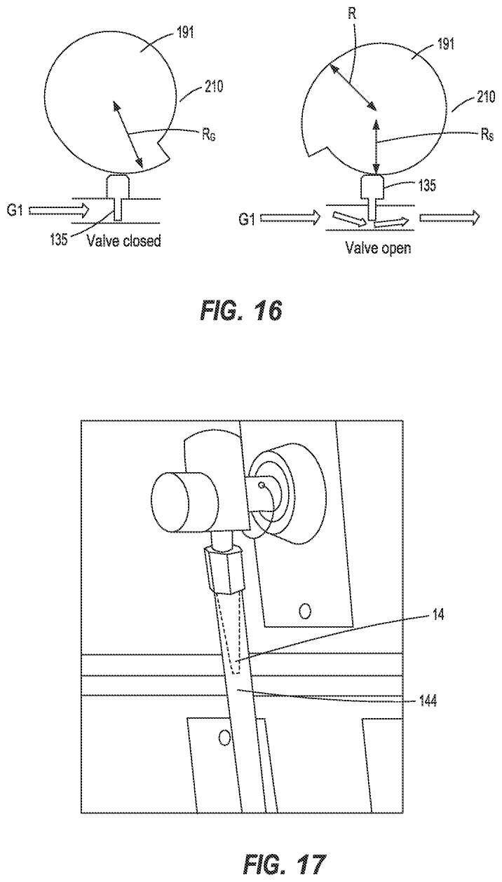

FIG. 16 provides a side view of a valve 135 in a “closed” position with a portion of cam gear 191 having a greater radius R_{G} being in contact with the valve 135, as shown on the left-hand side of FIG. 16. FIG. 16 also provides a side view of the same valve 135 in a “open” position with a portion of cam gear 191 having a smaller radius R_{S} being in contact with the valve 135, as shown on the right-hand side of FIG. 16.

Returning to FIG. 15, once a given valve 135 opens, gas flow G1 flows through the given valve 135, and applies a pneumatic pressure P_{O} onto either (1) a removable cartridge 130′ directly, or (ii) a pressure intensifier 500. In the particular embodiment shown in FIG. 15, a pneumatic pressure P_{O} is applied onto a pressure intensifier 500, which intensifies pneumatic pressure P_{O} to apply an intensified (e.g., an increased) pressure P_{I} onto a removable cartridge 130′, forcing a fragrance-carrying fluid 270 out of one or more nozzles 124′ into an atmosphere 200 outside the fragrance delivery device 100.

FIG. 18 illustrates an exemplary fragrance delivery device 100 of the present invention in combination with a compressed gas container 600 containing a compressed gas, which may or may not be in liquid or solid or supercritical form. Exemplary gasses include, but are not limited to, nitrous oxide, carbon dioxide, butane, propane, freons such as R-22, HFA gases such as HFA 134a or HFA 227, compressed air, compressed argon, compressed nitrogen, and the like. A regulator 601 may be used to reduce the pressure of the compressed gas container 600 to the working pressure of the fragrance delivery device 100. The gas supply pressure may be adjusted to the working pressure using the adjusting valve 603, and confirmed using the pressure gauge 604. The gas supplied through the regulator 601 exits through adaptor 602 and flows through plastic tubing 144 into the fragrance delivery device 100. Optionally, adjacent to the fragrance delivery device 100 mounted on the wall is a cartridge 130/130′ refill dispenser 138.

Compressed gas suitable for use in the fragrance dispensing devices 100 of the present invention may be purchased in the form of compressed gas cylinders/containers 600. High pressure gasses may be sold in cylindrical tanks 600 made from steel, aluminum, or carbon fiber composites. Lower pressure gases (e.g. HFA134a, FREON® gasses, such as R22, and the like) may be sold in cylindrical containers (not shown) similar to common household aerosol cans. For instance, Office Depot® branded “Cleaning Duster” is used to blow dust and debris from electronics and other office equipment, and contains 284 g of HFA 134a gas in an aerosol can format. If compressed gas cylinders 600 are used, multiple gas options are commercially available. Hydrofluoroalkanes (HFA)s such as HFA 134a (also known as 1,1,1,2-tetrafluoroethane, norfluorane, sold as SOLKANE® 134a by Diakin Refrigerants, Europe,) or HFA 227 (also known as 1,1,1,2,3,3,3-heptafluoropropane, apafluorane, sold as SOLKANE® 227 by Daikin) may be used to power the fragrance dispensing devices 100 and disperse the odor neutralizing or odor absorbing material into the air. If HFA 227 is used, the headspace pressure (the pressure above the liquified gas) is close to 80 psi at room temperature, and so no regulator 601 is needed between the gas cylinder 600 and the fragrance dispensing devices 100.

FREON® gasses, such as R-22, are physically similar to HFA gasses, however they happen to be chlorofluorocarbon (CFC) gases. While these would work to power the fragrance dispensing devices 100 mechanically, they are most undesirable due to their ability to destroy the protective ozone layer of the upper atmosphere.

Other gases that may be used include carbon dioxide, compressed air, nitrous oxide, nitrogen, propane, and butane. The gas supply line may include one or more gas regulators 601 to downgrade the pressure to a working lower pressure close to 55 psi, and optionally a pressure gauge 604 to indicate when the gas cylinder 600 or gas supply is depleted.

Gases that can be liquified and/or solidified offer the advantage of a phase transition from solid or liquid to gas as the gas is consumed. This gives two advantages: (a) constant pressure as the delivered to the regulator 601 as the gas vaporizes and (b) a large change in volume from solid/liquid to gas.

The gas from the compressed gas supply line may be used at the head space pressure. For instance, the head space in a compressed carbon dioxide cylinder 600 is approximately 800 psi at room temperature. This pressure may be enough to drive a piston/ball 411/431 and force atomization of a liquid into the air without using a pressure intensifier 500. Alternatively, a pressure intensifier 500 may be used which has a lower intensification ration than when using 55 psi medical air, to upgrade the final pressure within the liquid cylinder 600 from 800 psi to above around 1,000 psi.

Various different gas cylinders 600 may be used in connection with the fragrance dispensing devices 100. In one embodiment, a gas cylinder 600 is fitted with a first regulator 601, connected to a hose (not shown). The hose is connected to the fragrance dispensing device 100 via an optional factory set second pressure regulator housed within the casing (not shown). From the optional second pressure regulator (not shown), the gas line may optionally be connected to a tee-fitting (not shown). To one branch of the tee, a pressure gauge (not shown) may be connected that indicates to the user whether the gas pressure is sufficient for the device to be actuated correctly. The other branch of the tee (not shown) may be connected to the air inlet (not shown) for the timing device (not shown).

Connection of the gas cylinder 600 may be achieved via various means. The gas cylinder 600 may include an on-off valve 603, either a ball valve, or a gate valve for example, operated by turning a lever or rotating a knob. The outlet may be threaded to allow connection of a hose, or a regulator 601, and or a pressure gauge 604. Alternatively, the gas cylinder 600 may terminate in some kind of push to fit valving (not shown), which opens when a nipple (not shown) is inserted and closes when it is removed. Such devices include industrial interchange brass/steel quick-connect air coupling plug kits, for example Amflo Quick Connect Hose Coupling model number 12-201, sold by Granger Supply, or Type M Air Coupler sets, sold by Eastwood Company.

Suppliers of connections such as these are numerous, such as Colorfit by Milton M-Style Coupler and Plug kit sold by Milton Industries, Chicago IL. Plastic push-to-connect straight (or elbow) union tube fittings (not shown) may also be used to connect airlines, both within the device as well as from the compressed air or other gas tank 600 to the device. Such fittings are made and sold by many different manufacturers and vendors, including for example PneumaticPlus, based in Carson CA. Using such fittings is convenient for assembly, as the plastic tubing 144 is simply pushed into the plastic push-to-connect union tube fitting and is held in place by grips within the fitting.

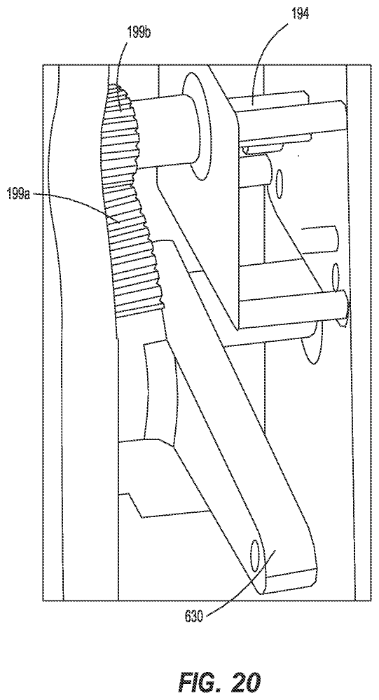

FIG. 19 illustrates a cross-sectional view of an exemplary cam shaft 195 that could be an alternative to the cam shaft 195 shown in FIGS. 9-10. As shown in FIG. 19, cam shaft 195 is used in combination with ball bearings 620, such as a ball bearing contained within two concentric rings, and tubular spacers 621. These tubular spacers 621 are not firmly connected to the cam shaft 195; however, spaced apart laterally along the cam shaft 195, and ensure that they do not move laterally along the shaft axis. In this embodiment, cam gears 191 are connected to spur gear 196, which is driven by the cam shaft 195. The rewind spring connector 197a is fixed directly to the cam shaft 195, which drives the spur gear 196.

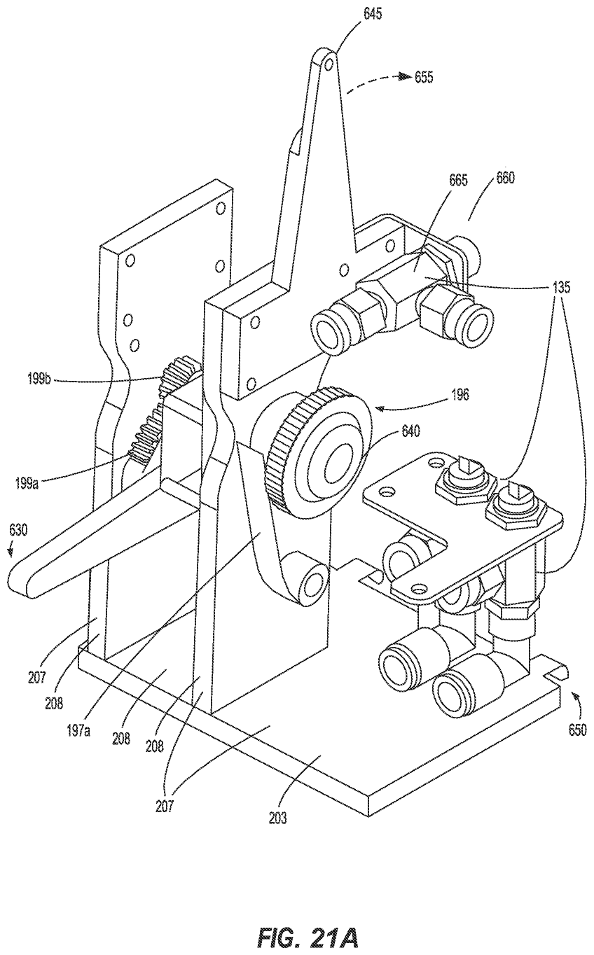

FIG. 20 illustrates an alternate embodiment in which the rack-n-pinion assembly 199 shown in FIGS. 9-10 is replaced with a gear 199A attached to a lever 630. The lever 630 in turn drives gear 199b, which attaches to the driver shaft 194, and connects to the spur gears 196.

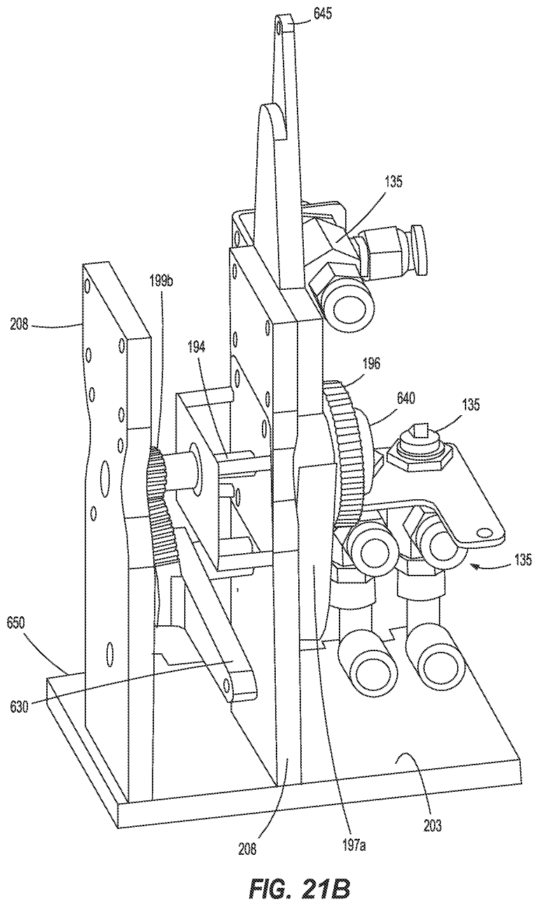

FIGS. 21A-21B depict two different perspective views of some of the fragrance device components within another assembly of mechanical components 700 used to form an alternate fragrance device 100 according to the present invention. As shown in FIGS. 21A-21B, the rack-n-pinion rewinding mechanism/assembly 199 is replaced by a lever 630 connected to a toothed gear 199a, which couple with a second gear 199b. Gear 199b drives shaft 194, which winds the spring within the timer (not shown) and through a one-way clutch 640, which does not turn the spur gear 196 while the timer is being wound, but does allow rotation of spur gear 196 when the timer is unwinding. Thus, winding of the timing mechanism spring does not impact the rotation of the cam gears 191, which operate two of the valves 135. Projection 645 connects to a mechanism (not shown) that moves the assembly of mechanical components 700 in the rear direction 655 when a removable cartridge 130′ is inserted into the deodorizing device 100. Motion is facilitated by a hinge 650, one half of which is shown, that allows the assembly of mechanical components 700 to rock back and forth. As the mechanism rocks back the activating button 660 on the normally closed air supply valve 665 interacts with a second part of the deodorizing device 100 (not shown) to provide compressed air power to the remaining two valves. When the removable cartridge 130′ is ejected, the assembly of mechanical components 700 rocks in the reverse direction of 655 and valve 665 shuts off the air supply.

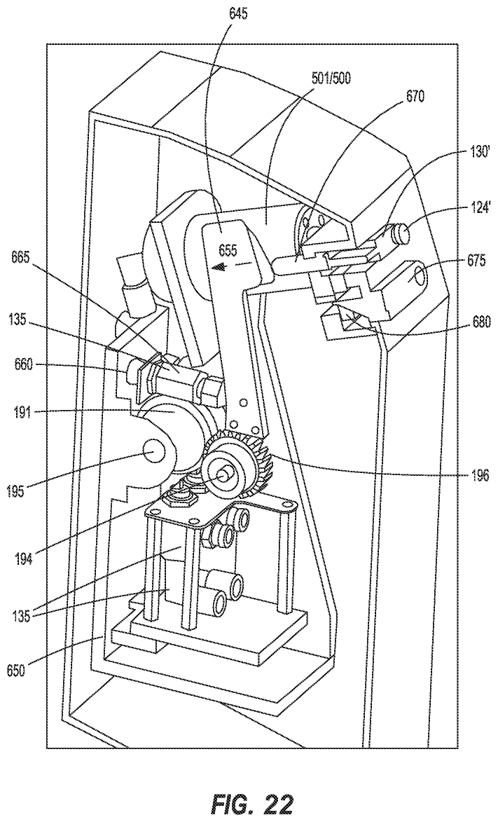

FIG. 22 depicts a cut-away view of an exemplary fragrance device 100 according to the present invention with a removable cartridge 130′ installed therein. As shown in FIG. 22, a portion of a pressure intensifier 500 is also shown. When removable cartridge 130′ is inserted and pushed home, latch mechanism 680 latches the removable cartridge 130′ in place. Communication pin 670 pushes against projection 645, which rocks the timing mechanism in direction 655 around the hinge 650. Among other actions, this motion depresses the actuator button 660 on the air supply valve 665. Moving the assembly in the direction 655 also engages the spur gear 196 with the cam shaft 195 spur gear (not shown). When the cartridge eject button 675 is depressed, then latch 680 releases removable cartridge 130′. Communication pin 670 retracts and projection 645 moves in the opposite direction to direction 655. This motion releases the button 660 on air valve 665, shutting off the air supply to the other valves, and disengages the spur gears 196, allowing the cam shaft 195 to freely rotate under the force of the rewind spring 197a (not shown in FIG. 22).

The timing mechanism 190 within the device 100 may be driven by clockwork mechanisms as described. Alternatively, an electrical programmable logic controller (PLC) (not shown) may be used to operate electrical solenoids (not shown) to open and close valves and trigger the pressure intensifier 500. Medical grade batteries and cells (not shown) are available and may be used to power timing logic within the device 100. As a further option, the PLC may be powered using 110 V AC outlet voltage or 240 V AC or other municipal supply.

If further non-electrical versions of the timing mechanism 190 are sought as alternatives to clockwork, then pneumatic logic controllers (not shown) may also be used. Pneumatic logic controllers were used years ago before electrically powered programmable logic controllers were invented. Pneumatic valves may be configured in such a way to time and control deployment of the pressure intensifiers 500 as needed. Pneumatic logic circuits perform various combinational and sequential logic calculations with binary pneumatic signals (atmosphere and vacuum), producing cascadable outputs based on Boolean operations. Such devices are described in numerous academic papers, for example in: Mingsoung Rhee & Mark A. Burns, Lab Chip. 2009 Nov. 7; 9(21): 3131-3143. doi:10.1039/b904354c. Medical compressed air is an ideal power source for pneumatic logic because it is conditioned, cleaned, regulated, and has a low particle count. As with clockwork mechanisms, pneumatic logic and timers do not require the use of electrical power.

Medical battery cells (such as D-size medical cells, sold as D MedCell alkaline batteries, sold by Medline) may also be used to power the fragrance delivery devices 100. They may be used to power electronic logic, which times the firing of spray from the fragrance delivery devices 100. Alternatively, batteries may be used to power a small compressor, sufficient to power the fragrance delivery devices 100.

Compressed gasses can also be used to power the timing mechanism instead of using a wound spring. The compressed gas may operate a pneumatic powered motor (not shown). Such motors are well known, and commonly used in environments where electricity may cause problems, such as in the presence of explosive vapors. Pneumatic motors may work in a number of different ways. The motor may contain a turbine structure that is rotated as air passes through. Alternatively, a motor may use reciprocating pistons to drive a circular motion. Piston and turbine motors may be purchased from Atlas Copco Group for example, who supply both turbine and piston driven pneumatic motors. A pneumatic motor may be used to power the timing cams 191, which in turn operate air valves 135/665 that fire the pressure intensifiers 500.

It is expected that fragrance delivery devices 100 according to the present invention may provide efficient means for avoiding, or otherwise removing, unpleasant odors, particularly in healthcare facilities, with a simple “plug-in and use” functionality with an on-site compressed gas source 20. These fragrance delivery devices 100 may be formed as single or multiple use products that are easily manufactured and made available in bulk supply for use at healthcare facilities (or any other setting) in all settings from emergency rooms and major trauma units to small medical practice offices.

The fragrance delivery devices 100 of the present invention, methods of making fragrance delivery devices 100 of the present invention, and methods of using fragrance delivery devices 100 of the present invention, are further described in the following additional embodiments.

### Additional Embodiments

Fragrance Delivery Devices

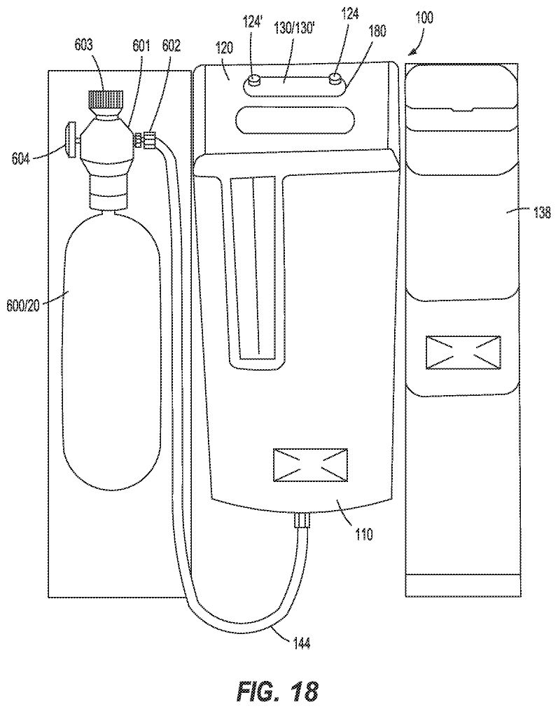

1. A fragrance delivery device 100 comprising: a body 101 defining an interior space 112, the body 101 comprising a nozzle coupler 122 designed to be attachable to a source of compressed gas, such as an existing air supply line 14 of a room, such as a patient care room, or a compressed gas container 600, the fragrance delivery device 100 being adapted to utilize pneumatic pressure P_{O} of a first gas flow G1 from the source of compressed gas 20, such a compressed gas cylinder 600 or the existing air supply line 12, to disperse and/or dispense a fragrance-carrying fluid 270 into an atmosphere 200 outside the fragrance delivery device 100.
2. The fragrance delivery device 100 according to embodiment 1, wherein the pneumatic pressure P_{O} of the source of compressed gas 20, such a compressed gas cylinder 600 or the existing air supply line 12 is used to force the fragrance-carrying fluid 270 from the fragrance delivery device 10 into the atmosphere 200 outside the fragrance delivery device 100.
3. The fragrance delivery device 100 according to embodiment 2, wherein the pneumatic pressure P_{O} of the source of compressed gas 20, such as a compressed gas cylinder 600 or the existing air supply line 12 is used to (1) generate an intensified (e.g., an increased) pressure P_{I}, and (2) utilize the intensified pressure P_{I} (e.g., an increased hydraulic pressure P_{I}) to disperse a fragrance-carrying fluid 270 into the atmosphere 200 outside the fragrance delivery device 100.
4. The fragrance delivery device 100 according to any one of embodiments 1 to 3, wherein the fragrance delivery device 100 disperses the fragrance-carrying fluid 270 into the atmosphere 200 outside the fragrance delivery device 100 without using any internal or external electric power to disperse the fragrance-carrying fluid 270. In other words, the control of whether the fragrance delivery device 100 is “on” (i.e., dispersing and/or dispensing) or “off” (i.e., not dispersing and/or dispensing) does not require any electronic components, and is accomplished by an assembly of mechanical components, such as exemplary assembly of mechanical components 700 detailed below. Body 101 of the fragrance delivery device 100 is only connected to the source of compressed gas 20, such as a compressed gas cylinder 600 or an existing air supply line 12 of a patient care room (or any other room), and not connected to any other device or electrical outlet in the patient care room (or the other room).
5. The fragrance delivery device 100 according to any one of embodiments 1 to 4, wherein the fragrance delivery device 100 comprises an assembly of mechanical components 700, said assembly of mechanical components 700 comprises one or more of: (a) a timing mechanism 190 that runs for a desired length of time (e.g., from 2.0 seconds to 60 minutes, typically about a 30 minute duration while providing approximately 210 degrees of output shaft 193 rotation thru this 30 minute cycle); (b) an output shaft 193 extending from the timing mechanism 190, and rotating for a desired length of time (e.g., from 2.0 seconds to 60 minutes)(see, FIGS. 9-10); (c) a driver shaft 194 that is axially connected to the output shaft 193 (see, FIGS. 9-10); (d) a cam shaft 195, which is preferably in an “offset” position relative to the driver shaft 194 (see, FIGS. 9-10); (e) a combination of spur gears 196 that connect the driver shaft 194 to the cam shaft 195 (e.g., the cam shaft 195 is “disconnected” from the driver shaft 194 when the combination of spur gears 196 are separated from one another, which allows a coil spring assembly 197 to rotate the cam shaft 195 back to a “reset/start” position; when the driver shaft 194 is “connected” to the cam shaft 195, the cam shaft 195 also rotates for a desired length of time (e.g., from 2.0 seconds to 60 seconds))(see, FIGS. 9-10); (f) one or more cam gears 191 designed to (i) rotate when the cam shaft 195 rotates, and (i) open and close one or more valves 135 during rotation (see, FIGS. 9-10); (g) the one or more valves 135 that control when pneumatic pressure is applied to disperse a fragrance-carrying fluid 270 (not shown in FIG. 8)(see, FIGS. 9-10) into the atmosphere 200 outside the fragrance delivery device 100; (h) an air supply valve 134 that controls air flow of a first gas flow G1 from the source of compressed gas 20, such as a compressed gas cylinder 600 or an existing air supply line 12; (i) a coil spring assembly 197 designed to rotate the cam shaft 195 back to a “reset/start” position following a dispersion step, the coil spring assembly 197 comprising (h)(i) a cam shaft reset wheel 197a, and (h)(ii) a coiled spring 197b; (j) a driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198 (see, FIGS. 9-10); (k) a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows the pinon gear 199b to spin in an opposite unwind direction along gear strip 199C (see, FIGS. 9-10); (l) a lever 630 connected to a toothed gear 199a, which couples with a second gear 199b that drives shaft 194, which winds a spring 197b within the timer 190 and through a one-way clutch 640, which does not turn a spur gear 196 while the timer 190 is being wound, but does allow rotation of spur gear 196 when the timer 190 is unwinding; (m) an adjustable stop pin 201 that provides a correct rotation start position for the cam shaft 195 (see, FIG. 9); and (n) one or more pressure intensifiers 500, each pressure intensifier 500 being capable of generating an intensified (e.g., an increased) pressure used to disperse fragrance-carrying fluid 270 into an atmosphere 200 outside the fragrance delivery device 100.
6. The fragrance delivery device 100 according to any one of embodiments 1 to 5, wherein the fragrance delivery device 100 comprises a timing mechanism 190 that runs for a desired length of time ranging from about 2.0 seconds (sec) to about 60 minutes (min). Typically, the timing mechanism 190 runs for a desired length of time ranging from about 5.0 (sec) to about 60.0 (min) (or any desired time period between about 5.0 sec to about 60.0 min, in intervals of 1.0 sec, e.g., 150 sec, or any range between about 5.0 sec to about 60.0 min, in intervals of 1.0 sec, e.g., from about 300 sec to about 30 min). In some embodiments, timing mechanism 190 is purely mechanical, i.e., does not run on nor depend on electricity to run. In other embodiments, timing mechanism 190 may require battery power or another source of electricity to operate.
7. The fragrance delivery device 100 according to embodiment 5 or 6, wherein the timing mechanism 190, when initiated, runs for about 5.0 min to about a 45 min.
8. The fragrance delivery device 100 according to any one of embodiments 5 to 7, wherein the timing mechanism 190, when initiated, runs for about 30 min.
9. The fragrance delivery device 100 according to any one of embodiments 5 to 8, wherein said assembly of mechanical components 700 comprises an output shaft 193 extending from the timing mechanism 190, and rotating for the desired length of time.
10. The fragrance delivery device 100 according to any one of embodiments 5 to 9, wherein said assembly of mechanical components 700 comprises a driver shaft 194 that is axially connected to the output shaft 193 (see, FIGS. 9-10), and rotates for the desired length of time.
11. The fragrance delivery device 100 according to any one of embodiments 5 to 10, wherein said assembly of mechanical components 700 comprises a cam shaft 195, said cam shaft 195 being engageable with and disengageable with the driver shaft 194 (see, FIGS. 9-10).
12. The fragrance delivery device 100 according to embodiment 11, wherein the cam shaft 195 is in an “offset” position relative to the driver shaft 194 (see, FIGS. 9-10).
13. The fragrance delivery device 100 according to any one of embodiments 5 to 12, wherein said assembly of mechanical components 700 comprises a combination of spur gears 196 that connect the driver shaft 194 to the cam shaft 195.
14. The fragrance delivery device 100 according to embodiment 13, wherein the cam shaft 195 is “disconnected” from the driver shaft 194 when two or more gears within the combination of spur gears 196 are separated from one another.
15. The fragrance delivery device 100 according to any one of embodiments 5 to 14, wherein when the driver shaft 194 is “connected” to the cam shaft 195, the cam shaft 195 also rotates for the desired length of time (e.g., from 2.0 sec to 60 min that the output shaft 193 and the driver shaft 194 rotate when the timing mechanism 190 is activated)(see again, FIGS. 9-10).
16. The fragrance delivery device 100 according to any one of embodiments 5 to 15, wherein said assembly of mechanical components 700 comprises one or more cam gears 191 designed to (i) rotate when the cam shaft 195 rotates, and (i) open and close one or more valves 135 during rotation (see, FIGS. 9-10).
17. The fragrance delivery device 100 according to any one of embodiments 5 to 16, wherein each cam gear 191 of said one or more cam gears 191 independently has a radius R that varies along a periphery 210 of the cam gear 191.
18. The fragrance delivery device 100 according to any one of embodiments 5 to 17, wherein each cam gear 191 of said one or more cam gears 191 independently has a peripheral shape so as to result in one or more times that the one or more valves 135 are opened during a rotation of the cam gear 191. For example, in some cases, a given cam gear 191 may have a peripheral shape that results in the one or more valves 135 opening and closing a single time during rotation of the given cam gear 191. See, for example, the exemplary cam gear 191 shown in FIGS. 15A-16. In other cases, a given cam gear 191 may have a peripheral shape that results in the one or more valves 135 opening and closing two or more times during rotation of the given cam gear 191. In these cases, the cam gear 191 could have two or more sections along the periphery 210 of the cam gear 191 wherein the radius R_{S} is less than a radius R_{G} on either side of each section (i.e., each section with the smaller radius R_{S}).
19. The fragrance delivery device 100 according to any one of embodiments 5 to 18, wherein each cam gear 191 of said one or more cam gears 191 independently rotates up to 360° of rotation once the timing mechanism 190 is activated. It should be understood that each cam gear 191 of said one or more cam gears 191 may independently rotate any desired amount up to 360° of rotation or greater once the timing mechanism 190 is activated. Typically, each cam gear 191 of said one or more cam gears 191 independently rotates from about 45° to about 270° of rotation once the timing mechanism 190 is activated (or any value between about 45° and about 270°, in 1° increments, e.g., 255°, or any range of values between about 45° and about 270°, in 1° increments, e.g., from about 180° to about 270°).
20. The fragrance delivery device 100 according to any one of embodiments 5 to 19, wherein each cam gear 191 of said one or more cam gears 191 independently rotates from about 180° to about 270° of rotation once the timing mechanism 190 is activated.
21. The fragrance delivery device 100 according to any one of embodiments 5 to 20, wherein said assembly of mechanical components 700 comprises the one or more valves 135 that control when pneumatic pressure P_{O} is applied so as to disperse a fragrance-carrying fluid 270 into the atmosphere 200 outside the fragrance delivery device 100.
22. The fragrance delivery device 100 according to any one of embodiments 5 to 21, wherein the one or more valves 135 comprises two or more valves 135.
23. The fragrance delivery device 100 according to any one of embodiments 5 to 22, wherein the one or more valves 135 comprises two valves 135.
24. The fragrance delivery device 100 according to any one of embodiments 5 to 23, wherein said assembly of mechanical components 700 comprises an air supply valve 134 that controls air flow of a first gas flow G1 from the source of compressed gas 20, such as an existing air supply line 12 or alternatively a compressed gas container 600.
25. The fragrance delivery device 100 according to any one of embodiments 5 to 24, wherein said assembly of mechanical components 700 comprises a coil spring assembly 197 designed to rotate the cam shaft 195 back to a “reset/start” position following a dispersion step.
26. The fragrance delivery device 100 according to any one of embodiments 5 to 25, wherein the coil spring assembly 197 comprises (i) a cam shaft reset wheel 197a, and (ii) a coiled spring 197b.
27. The fragrance delivery device 100 according to any one of embodiments 5 to 26, wherein said assembly of mechanical components 700 comprises a driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198 (see, FIGS. 9-10).
28. The fragrance delivery device 100 according to any one of embodiments 5 to 27, wherein said assembly of mechanical components 700 comprises a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows a pinon gear 199b to spin in an opposite unwind direction along gear strip 199C (see, FIGS. 9-10).
29. The fragrance delivery device 100 according to any one of embodiments 5 to 27, wherein said assembly of mechanical components 700 comprises a lever 630 connected to a toothed gear 199a, which couples with a second gear 199b that drives shaft 194, which winds a spring 197b within the timer 190 and through a one-way clutch 640, which does not turn a spur gear 196 while the timer 190 is being wound, but does allow rotation of spur gear 196 when the timer 190 is unwinding. See, for example, FIGS. 21A-21B.
30. The fragrance delivery device 100 according to any one of embodiments 5 to 29, wherein said assembly of mechanical components 700 comprises an adjustable stop pin 201 that provides a rotation start position for the cam shaft 195 (see, FIG. 9).
31. The fragrance delivery device 100 according to any one of embodiments 5 to 30, wherein said assembly of mechanical components 700 comprises one or more pressure intensifiers 500, each pressure intensifier 500 being capable of generating an intensified (e.g., an increased) pressure P_{I} used to disperse fragrance-carrying fluid 270 into the atmosphere 200 outside the fragrance delivery device 100.
32. The fragrance delivery device 100 according to any one of embodiments 5 to 31, wherein each pressure intensifier 500 has a pneumatic pressure intensifier air inlet coupling member 509 that enables an adapter 144 (e.g., plastic tubing) to connect (i) the air inlet coupling member 509 to (ii) the pneumatic pressure P_{O} of the source of compressed gas 20, such as a compressed gas cylinder 600 or the existing air supply line 12. See, for example, an exemplary adapter 144 in the form of plastic tubing shown in FIGS. 17-18.
33. The fragrance delivery device 100 according to any one of embodiments 5 to 32, wherein said assembly of mechanical components 700 comprises one or more air line connections 206, wherein the one or more air line connections 206 provide one or more air inlets (i.e., supply) and one or more air outlets (i.e., exhaust) for the first gas flow G1 to move to and from the one or more valves 135 (e.g., two air line connections 206 per side as shown in FIGS. 8, 10, and 11).
34. The fragrance delivery device 100 according to embodiment 33, wherein two or more air line connections 206 within the one or more air line connections 206 are in fluid communication with one another.
35. The fragrance delivery device 100 according to embodiment 33 or 34, wherein the one or more air line connections 206 comprise (1) an air inlet 206′ for the first gas flow G1 to move towards the one or more valves 135 (e.g., see the left-hand side of one of two vertically-extending pivot plates 208 of a pivoting plate assembly 207 shown in FIG. 10), and (2) an air outlet 206″ for the first gas flow G1 to move away from the one or more valves 135 (e.g., see the right-hand side of the other vertically-extending pivot plate 208 of the exemplary pivoting plate assembly 207).
36. The fragrance delivery device 100 according to embodiment 35, wherein the air outlet 206″ provides the pneumatic pressure P_{O} of first gas flow G1 from the source of compressed gas 20, such as a compressed gas cylinder 600 or the existing air supply line 12 so as to disperse the fragrance-carrying fluid 270 into the atmosphere 200 outside the fragrance delivery device 100.
37. The fragrance delivery device 100 according to embodiment 35 or 36, wherein an adapter 144 (e.g., plastic tubing, not shown) connects (i) the air outlet 206″ to (ii) a fragrance medium container 130 housing a fragrance medium 170.
38. The fragrance delivery device 100 according to any one of embodiments 35 to 37, wherein an adapter 144 (e.g., plastic tubing, not shown) connects (i) the air outlet 206″ to (ii) an air inlet coupling member 509 of a pressure intensifier 500. See, for example, FIG. 8.
39. The fragrance delivery device 100 according to any one of embodiments 35 to 38, wherein the air inlet 206′ is provided the first gas flow G1 from the air supply valve 134 from air supply line 12 or compressed gas cylinder/container 600.
40. The fragrance delivery device 100 according to embodiment 39, wherein a second adapter 144 (e.g., plastic tubing, not shown) connects (i) the air inlet 206′ to (ii) the air supply valve 134 from gas supply 12.
41. The fragrance delivery device 100 according to any one of embodiments 5 to 40, wherein said assembly of mechanical components 700 comprises all components shown in embodiment 5 except for (1) the one or more pressure intensifiers 500, and (2) one of (k) or (l).
42. The fragrance delivery device 100 according to any one of embodiments 5 to 40, wherein said assembly of mechanical components 700 comprises all components shown in embodiment 5 including the one or more pressure intensifiers 500 except for either (k) or (l).
43. The fragrance delivery device 100 according to any one of embodiments 5 to 40 and 42, wherein each pressure intensifier 500 comprises one or more of the following components: (1) a master cylinder side wall 501, (2) a bottom end cap 502, (3) a top end cap 503, (4) a top end cap slot 504 within an outer surface 511 of the top end cap 503, the top end cap slot 504 being sized to accept at least a portion of an impact surface end 132 of a given removable cartridge 130′, (5) a pneumatic pressure intensifier air inlet coupling member 509, (6) a bottom end cap air channel 505, (7) a pneumatic pressure intensifier piston 506, (8) a connecting rod 507 attached to the pneumatic pressure intensifier piston 506, (9) a return spring 508 positioned on and encircling at least a portion of the connecting rod 507, (10) a top end cap channel 510 within an inner surface 512 of the top end cap 503, the top end cap channel 510 being sized to receive a connecting rod impact end 513 of the connecting rod 507, and (11) a master cylinder volume 515 bound by an inner surface 517 of the master cylinder side wall 501, a bottom end cap inner surface 514 of the bottom end cap 502, and the top end cap 503, and the inner surface 512 of the top end cap 503. See, for example, FIG. 13.
44. The fragrance delivery device 100 according to any one of embodiments 5 to 40 and 42 to 43, wherein each pressure intensifier 500 comprises each of: (1) a master cylinder side wall 501, (2) a bottom end cap 502, (3) a top end cap 503, (4) a top end cap slot 504 within an outer surface 511 of the top end cap 503, the top end cap slot 504 being sized to accept at least a portion of an impact surface end 132 of a given removable cartridge 130′, (5) a pneumatic pressure intensifier air inlet coupling member 509, (6) a bottom end cap air channel 505, (7) a pneumatic pressure intensifier piston 506, (8) a connecting rod 507 attached to the pneumatic pressure intensifier piston 506, (9) a return spring 508 positioned on and encircling at least a portion of the connecting rod 507, (10) a top end cap channel 510 within an inner surface 512 of the top end cap 503, the top end cap channel 510 being sized to receive a connecting rod impact end 513 of the connecting rod 507, and (11) a master cylinder volume 515 bound by an inner surface 517 of the master cylinder side wall 501, a bottom end cap inner surface 514 of the bottom end cap 502, and the top end cap 503, and the inner surface 512 of the top end cap 503.
45. The fragrance delivery device 100 according to embodiment 43 or 44, wherein the pneumatic pressure intensifier piston 506 has a piston surface area A_{PO} that is greater than a piston surface area A_{PC} of the cartridge piston 411 of a removable cartridge 130′.
46. The fragrance delivery device 100 according to any one of embodiments 43 to 45, wherein the pneumatic pressure intensifier piston 506 has a piston surface area A_{PO} that provides an intensified (e.g., an increased) pressure P_{I} that is from about 2 to 24 times greater than the pneumatic pressure P_{O} of the first gas flow G1 from the source of compressed gas 20, such as a compressed gas cylinder/container 600 or the existing air supply line 12.
47. The fragrance delivery device 100 according to any one of embodiments 5 to 40 and 42 to 46, wherein each pressure intensifier 500 further comprises: one or more o-ring grooves 554 that circumscribe an outer side surface 555 of a pressure intensifier piston 506 positioned within a master cylinder volume 515 of a given pressure intensifier 500, each of the one or more o-ring grooves 554 being sized to contain one or more o-rings 552. See, for example, FIGS. 13A-13B.
48. The fragrance delivery device 100 according to embodiment 47, wherein each of the one or more o-ring grooves 554 is designed to (1) apply a sealing force onto one or more o-rings 552 positioned within a given o-ring groove 554, the sealing force forcing the one or more o-rings 552 between (i) an inner surface 517 of the master cylinder volume 515 and (ii) an outer side surface 555 of the pressure intensifier piston 506 while the pressure intensifier piston 506 is pushed by the pneumatic pressure P_{O} of the first gas flow G1, and (2) apply a minimal force, if any, onto the one or more o-rings 552 positioned within a given o-ring groove 554 and between (i) an inner surface 517 of the master cylinder volume 515 and (ii) an outer side surface 555 of the pressure intensifier piston 506 while the pressure intensifier piston 506 retreats back into an initial position once the pneumatic pressure P_{O} of the first gas flow G1 is stopped. See again, for example, FIGS. 13A-13B.
49. The fragrance delivery device 100 according to embodiment 47 or 48, wherein the pressure intensifier piston 506 comprises one or more o-ring grooves 554 comprises two or more o-ring grooves 554 that each circumscribe an outer side surface 555 of a pressure intensifier piston 506 positioned within a master cylinder volume 515 of a given pressure intensifier 500.
50. The fragrance delivery device 100 according to any one of embodiments 47 to 49, further comprising one or more o-rings 552 positioned within each of the one or more o-ring grooves 554.
51. The fragrance delivery device 100 according to any one of embodiments 47 to 50, further comprising two or more o-rings 552 positioned within each of the one or more o-ring grooves 554.
52. The fragrance delivery device 100 according to any one of embodiments 5 to 51, wherein the components within the exemplary assembly of mechanical components 700 are either (1) connected to (directly or indirectly) a fixed (i.e., stationary) vertical base plate 202, attached to a fixed (i.e., stationary) horizontal base plate 204, or (2) positioned on and connected to a pivoting plate 203, the pivoting plate 203 being connected, via a hinge 205, to the fixed vertical base plate 202 (see, FIGS. 8 and 11).
53. The fragrance delivery device 100 according to embodiment 52, wherein a position of the pivoting plate 203 is controlled with an activation lever or button (e.g., activation lever 192) positioned along an outer surface of an outer casing 110 of the fragrance delivery device 100.
54. The fragrance delivery device 100 according to embodiment 52 or 53, wherein a position of the pivoting plate 203 is controlled with an activation lever 192.
55. The fragrance delivery device 100 according to embodiment 52, wherein a position of the pivoting plate 203 is controlled by the presence or absence of a removable cartridge 130′ positioned within a removable cartridge port 180 of the fragrance delivery device 100.
56. The fragrance delivery device 100 according to any one of embodiments 47 to 55, wherein when the pivoting plate 203 is in a horizontal position, the exemplary assembly of mechanical components 700 is “active” and (1) a main pneumatic pressure P_{O} from the source of compressed gas 20, such as a compressed gas cylinder/container 600 or the existing air supply 14 is on (e.g., air supply valve 134 from the source of compressed gas 20, such as a compressed gas cylinder/container 600 or the existing air supply 14 is “open”), and (2) the combination of spur gears 196 that connect the driver shaft 194 to the cam shaft 195 are engaged (i.e., connected with one another).
57. The fragrance delivery device 100 according to any one of embodiments 47 to 52, wherein when the pivoting plate 203 is pivoted into a downward position, two things happen simultaneously, namely: (1) a main pneumatic pressure P_{O} from the source of compressed gas 20, such as a compressed gas cylinder/container 600 or the existing air supply 14 is off (e.g., air supply valve 134 from the source of compressed gas 20, such as a compressed gas cylinder/container 600 or the existing air supply 14 is “closed”), and (2) the combination of spur gears 196 that connect the driver shaft 194 to the cam shaft 195 is disengaged (i.e., not connected with one another).
58. The fragrance delivery device 100 according to any one of embodiments 54 to 57, wherein movement of the activation lever 192 in a downward path (or insertion of a removable cartridge 130′ into a removable cartridge port 180 of the fragrance delivery device 100) moves the pivoting plate 203 from a downward position into a horizontal position, wherein the exemplary assembly of mechanical components 700 is “active” and (1) a main pneumatic pressure P_{O} from the source of compressed gas 20, such as a compressed gas cylinder/container 600 or the existing air supply 14 is on (e.g., air supply valve 134 from the source of compressed gas 20, such as a compressed gas cylinder/container 600 or the existing air supply 14 is “open”), and (2) the combination of spur gears 196 that connect the driver shaft 194 to the cam shaft 195 are engaged (i.e., connected with one another).
59. The fragrance delivery device 100 according to any one of embodiments 54 to 58, wherein movement of the activation lever 192 in a downward path (or insertion of a removable cartridge 130′ into a removable cartridge port 180 of the fragrance delivery device 100) activates the timing mechanism 190, which starts rotation of the one or more cam gears 191.
60. The fragrance delivery device 100 according to any one of embodiments 47 to 59, wherein the exemplary assembly of mechanical components 700 comprises a pivoting plate assembly 207 that comprises the pivoting plate 203, and one or more vertically-extending pivot plates 208 connected to pivoting plate 203 so as to move with the pivoting plate 203.
61. The fragrance delivery device 100 according to embodiment 60, wherein the one or more vertically-extending pivot plates 208 provide support for one or more of: (a) the timing mechanism 190, (b) the output shaft 193, (c) the driver shaft 194, (d) at least one spur gear 196′ within the combination of spur gears 196, (e) the one or more valves 135, (f) the coil spring assembly 197, (g) the driver shaft clutch 198, (h) the rack-n-pinion rewind assembly 199, and (i) lever 630 connected to toothed gear 199a, coupled with second gear 199b, and one-way clutch 640.
62. The fragrance delivery device 100 according to embodiment 60 or 61, wherein the one or more vertically-extending pivot plates 208 provide support for each of: (a) the timing mechanism 190, (b) the output shaft 193, (c) the driver shaft 194, (d) at least one spur gear 196′ within the combination of spur gears 196, (e) the one or more valves 135, (f) the coil spring assembly 197, (g) the driver shaft clutch 198, and one of: (h) the rack-n-pinion rewind assembly 199, and (i) lever 630 connected to toothed gear 199a, coupled with second gear 199b, and one-way clutch 640.

Preferred Fragrance Delivery Devices

63. The fragrance delivery device 100 according to any one of embodiments 1 to 62, wherein the body 101 further comprises a removable cartridge port 180 sized to accept a removable cartridge 130′ containing a fragrance medium 170, and the pneumatic pressure P_{O} of the first gas flow G1 is used to disperse the fragrance-carrying fluid 270 from a removable cartridge 130′ positioned within the removable cartridge port 180, the removable cartridge 130′ comprising one or more cartridge nozzles 124′ sized to allow passage of the fragrance-carrying fluid 270 from the removable cartridge 130′ into the atmosphere 200 outside the fragrance delivery device 100.
64. A fragrance delivery device 100 comprising: a body 101 defining an interior space 112, the body 101 comprising (i) a nozzle coupler 122 designed to be attachable to a source of compressed gas 20, such as a compressed gas cylinder/container 600 or an existing air supply line 12 of a room, such as a patient care room, and (ii) a removable cartridge port 180 sized to accept a removable cartridge 130′ containing a fragrance medium 170, the nozzle coupler 122 being adapted to receive a first gas flow G1 from the source of compressed gas 20, such as the compressed gas cylinder/container 600 or the existing air supply line 12 and utilize a pneumatic pressure P_{O} of the first gas flow G1 to disperse a fragrance-carrying fluid 270 from a removable cartridge 130′ positioned within the removable cartridge port 180, the removable cartridge 130′ comprising one or more cartridge nozzles 124′ sized to allow passage of the fragrance-carrying fluid 270 from the removable cartridge 130′ into an atmosphere 200 outside the fragrance delivery device 100.
65. The fragrance delivery device 100 according to embodiment 63 or 64, wherein the pneumatic pressure of the first gas flow G1 from the source of compressed gas 20, such as a compressed gas cylinder/container 600 or the existing air supply line 12 pushes on a cartridge piston 411 or cartridge ball 431 of the removable cartridge 130′ so as to force the fragrance-carrying fluid 270 from the one or more cartridge nozzles 124′ of the removable cartridge 130′ into the atmosphere 200 outside the fragrance delivery device 100. See, for example, FIGS. 13 and 14B-14C.
66. The fragrance delivery device 100 according to any one of embodiments 63 to 65, wherein the removable cartridge 130′ houses an amount of the fragrance medium 170 so as to provide one or more bursts of the fragrance-carrying fluid 270 from one or more nozzles 124′ positioned along an outer surface 131 of the removable cartridge 130′.
67. The fragrance delivery device 100 according to any one of embodiments 63 to 66, wherein the removable cartridge 130′ houses an amount of the fragrance medium 170 so as to provide one burst of the fragrance-carrying fluid 270 from one or more cartridge nozzles 124′ positioned along an outer surface 131 of the removable cartridge 130′.
68. The fragrance delivery device 100 according to any one of embodiments 63 to 66, wherein the removable cartridge 130′ houses an amount of the fragrance medium 170 so as to provide multiple bursts of the fragrance-carrying fluid 270 from one or more cartridge nozzles 124′ positioned along an outer surface 131 of the removable cartridge 130′.
69. The fragrance delivery device 100 according to any one of embodiments 63 to 68, wherein each of the one or more bursts of the fragrance-carrying fluid 270 provides up to about 30 minutes of odor-fighting protection.
70. The fragrance delivery device 100 according to any one of embodiments 63 to 64 and 66 to 69, wherein the removable cartridge 130′ is altered by the pneumatic pressure P_{O} exerted on an impact surface end 132 of the removable cartridge 130′ by the first gas flow G1.
71. The fragrance delivery device 100 according to embodiment 70, wherein the impact surface 132 of the removable cartridge 130′ is opposite the one or more cartridge nozzles 124′ positioned along the outer surface 131 of the removable cartridge 130′.
72. The fragrance delivery device 100 according to embodiment 70 or 71, wherein the impact surface 132 of the removable cartridge 130′ comprises a cartridge piston 411 of the removable cartridge 130′. See again, for example, FIGS. 13 and 14B.
73. The fragrance delivery device 100 according to embodiment 70 or 71, wherein the impact surface 132 of the removable cartridge 130′ comprises a cartridge ball 431 of the removable cartridge 130′. See again, for example, FIG. 14C.
74. The fragrance delivery device 100 according to any one of embodiments 63 to 73, wherein the removable cartridge 130′ comprises a single-use removable cartridge 130′.
75. The fragrance delivery device 100 according to any one of embodiments 63 to 74, wherein the removable cartridge 130′ further comprises a frangible membrane 556 extending over impact surface end 132, the frangible membrane 556 being puncturable/penetrable by the connecting rod impact end 513 of the connecting rod 507.
76. The fragrance delivery device 100 according to any one of embodiments 63 to 75, wherein the fragrance delivery device 100 further comprises a removable cartridge locking member 133 that engages with a removable cartridge 130′ upon insertion of the removable cartridge 130′ into the removable cartridge port 180. In some embodiments, the removable cartridge locking member 133 comprises a sliding removable cartridge locking member 133 as shown in FIG. 4A.
77. The fragrance delivery device 100 according to any one of embodiments 63 to 76, wherein insertion of the removable cartridge 130′ into the removable cartridge port 180 initiates one or more of: (i) moves the pivoting plate 203 from a downward position into a horizontal position, wherein the exemplary assembly of mechanical components 700 is “active”, (ii) opens an air supply valve 134 to one or more valves 135 leading to one or more of the removable cartridges 130′, (iii) engages a timing mechanism 190 that runs for a desired length of time, and/or (iv) engages two or more spur gears 196 of the combination of spur gears 196 with one another. See, for example, FIGS. 4, 7, and 11.
78. The fragrance delivery device 100 according to any one of embodiments 63 to 77, wherein insertion of the removable cartridge 130′ into the removable cartridge port 180 initiates each of: (i) opens an air supply valve 134 from the source of compressed gas 20, such as the compressed gas cylinder/container 600 or the existing air supply line 12 to one or more valves 135 leading to one or more of the removable cartridges 130′, (ii) engages a timing mechanism 190 that runs for a desired length of time, and (iii) engages two or more spur gears 196 of the combination of spur gears 196 with one another.
79. The fragrance delivery device 100 according to any one of embodiments 63 to 78, wherein the fragrance delivery device 100 further comprises an activation lever 192 that, once engaged, initiates one or more of: (i) activating a timing mechanism 190, and (ii) opening an air supply valve 134 to supply the first gas flow G1 from the source of compressed gas 20, such as the compressed gas cylinder/container 600 or the existing air supply line 12 to one or more valves 135 leading to one or more of the removable cartridges 130′. See, for example, FIG. 4.
80. The fragrance delivery device 100 according to any one of embodiments 63 to 79, wherein the fragrance delivery device 100 further comprises an activation lever 192 that, once engaged, initiates each of: (i) activating a timing mechanism 190, and (ii) opening an air supply valve 134 to supply the first gas flow G1 from the source of compressed gas 20, such as the compressed gas cylinder/container 600 or the existing air supply line 12 to one or more valves 135 leading to one or more of the removable cartridges 130′.
81. The fragrance delivery device 100 according to any one of embodiments 63 to 80, wherein ejection of the removable cartridge 130′ from the removable cartridge port 180 initiates one or more of: (i) moves the pivoting plate 203 from a horizontal position into a downward position, wherein the exemplary assembly of mechanical components 700 is “inactive”, (ii) closes an air supply valve 134 from the source of compressed gas 20, such as the compressed gas cylinder/container 600 or the existing air supply line 12 to one or more valves 135 leading to one or more of the removable cartridges 130′, (iii) disengages a timing mechanism 190 that runs for a desired length of time, and (iv) disengages two or more spur gears 196 of the combination of spur gears 196 from one another.
82. The fragrance delivery device 100 according to any one of embodiments 63 to 81, wherein ejection of the removable cartridge 130′ from the removable cartridge port 180 initiates each of: (i) closes an air supply valve 134 from the source of compressed gas 20, such as the compressed gas cylinder/container 600 or the existing air supply line 12 to one or more valves 135 leading to one or more of the removable cartridges 130′, (ii) disengages a timing mechanism 190 that runs for a desired length of time, and (iii) disengages two or more spur gears 196 of the combination of spur gears 196 from one another.
83. The fragrance delivery device 100 according to any one of embodiments 63 to 82, wherein said removable cartridge 130′ comprises one or more of: (1) one or more separate cartridge pistons 411, one or more separate cartridge balls 431, one for each fragrance-carrying fluid reservoir 412 positioned within the removable cartridge 130′, (2) one or more separate fragrance-carrying fluid reservoirs 412, (3) one or more separate nozzle outlets 137, one for each cartridge nozzle 124′, (4) one or more separate cartridge impact surface members 413, one for each fragrance-carrying fluid reservoir 412 and each cartridge piston 411 or cartridge ball 431, and (5) one or more frangible membranes 556 positioned over each of the one or more separate cartridge impact surface members 413. As discussed above, it should be understood that a given removable cartridge 130′ may comprise a single fragrance-carrying fluid reservoir 412 positioned within the removable cartridge 130′, or may comprise two or more fragrance-carrying fluid reservoirs 412 positioned within the removable cartridge 130′. See, for example, exemplary removable cartridge 130′ shown in FIGS. 14A-14B.
84. The fragrance delivery device 100 according to any one of embodiments 63 to 83, wherein said removable cartridge 130′ comprises: (1) one or more separate cartridge pistons 411 or cartridge ball 431, one for each fragrance-carrying fluid reservoir 412 positioned within the removable cartridge 130′, (2) one or more separate fragrance-carrying fluid reservoirs 412, (3) one or more separate nozzle outlets 137, one for each cartridge nozzle 124′, and (4) one or more separate cartridge impact surface members 413, one for each fragrance-carrying fluid reservoir 412.
85. The fragrance delivery device 100 according to any one of embodiments 63 to 84, wherein said removable cartridge 130′ comprises: (1) two separate cartridge pistons 411 or cartridge balls 431, one for each fragrance-carrying fluid reservoir 412 positioned within the removable cartridge 130′, (2) two fragrance-carrying fluid reservoirs 412, (3) two separate nozzle outlets 137, one for each nozzle 124′, and (4) two separate cartridge impact surface members 413, one for each fragrance-carrying fluid reservoir 412.
86. The fragrance delivery device 100 according to any one of embodiments 63 to 85, wherein the fragrance delivery device 100 further comprises a removable cartridge dispenser 138 sized to house two or more removable cartridges 130′. See, for example, exemplary removable cartridge dispenser 138 shown in FIGS. 4-7, and FIG. 18.
87. The fragrance delivery device 100 according to embodiment 86, wherein the removable cartridge dispenser 138 is sized to house from about 10 to about 50 removable cartridges 130′ (or any desired number of removable cartridges 130′).
88. The fragrance delivery device 100 according to embodiment 86 or 87, wherein the removable cartridge dispenser 138 is attachable and removable from the body 101 of the fragrance delivery device 100. See, for example, FIGS. 4-6.
89. The fragrance delivery device 100 according to any one of embodiments 86 to 88, wherein the removable cartridge dispenser 138 is integrally attached to the body 101 of the fragrance delivery device 100 (e.g., the removable cartridge dispenser 138 is molded to the body 101 of the fragrance delivery device 100).
90. The fragrance delivery device 100 according to any one of embodiments 63 to 89, wherein the fragrance delivery device 100 comprises one or more features as recited in any one of embodiments 1 to 62.

Even More Preferred Fragrance Delivery Devices

91. A fragrance delivery device 100 comprising: a body 101 defining an interior space 112, the body 101 comprising a nozzle coupler 122 designed to be attachable to a source of compressed gas 20, such as a compressed gas cylinder or an existing air supply line 12 of a room, such as a patient care room, the fragrance delivery device 100 being adapted to (1) utilize pneumatic pressure P_{O} from the existing air supply line 12 to generate an intensified (e.g., an increased) pressure P_{I}, and (2) utilize the intensified pressure P_{I} to disperse a fragrance-carrying fluid 270 into an atmosphere 200 outside the fragrance delivery device 100.
92. The fragrance delivery device 100 according to embodiment 91, wherein the body 101 further comprises one or more pressure intensifiers 500, each pressure intensifier 500 being capable of generating the intensified (e.g., an increased) pressure P_{I} used to disperse the fragrance-carrying fluid 270 into an atmosphere 200 outside the fragrance delivery device 100. See, for example, FIGS. 8, 13, and 15.
93. The fragrance delivery device 100 according to embodiment 91 or 92, wherein the intensified pressure P_{I} is used to push on a cartridge piston 411 or a cartridge ball 431 of a removable cartridge 130′ so as to force fragrance-carrying fluid 270 from one or more cartridge nozzles 124′ of the removable cartridge 130′ into an atmosphere 200 outside the fragrance delivery device 100.
94. The fragrance delivery device 100 according to any one of embodiments 91 to 93, wherein the fragrance delivery device 100 further comprises one or more features as recited in any one of embodiments 1 to 90.

Other Alternative, Less Preferred Fragrance Delivery Devices

95. The fragrance delivery device 100 according to any one of embodiments 1 to 62, wherein the pneumatic pressure P_{O} from a source of compressed gas 20, such as a compressed gas cylinder or the existing air supply line 12 is intermixed with a fragrance medium 170 to form and then disperse a fragrance-carrying fluid 270 from the fragrance delivery device 100.
96. A fragrance delivery device 100 comprising: a body 101 defining an interior space 112, the body 101 comprising a nozzle coupler 122 designed to be attachable to a source of compressed gas 20, such as a compressed gas cylinder/container 600 or an existing air supply line 12 of a patient care room, the fragrance delivery device 100 being adapted to (1) receive a first gas flow G1 from the source of compressed gas 20, such as a compressed gas cylinder/container 600 or the existing air supply line 12, (2) mix the first gas flow G1 with a fragrance medium 170 within or along the body 101 to generate a fragrance-carrying second gas flow G2, and (3) disperse the fragrance-carrying second gas flow G2 to an atmosphere 200 outside the fragrance delivery device 100 without using any internal or external electric power (i.e., body 101 does not contain any electronic components and is not connected to any electronic components or sources of electricity; body 101 is only connected to the source of compressed gas 20, such as a compressed gas cylinder/container 600 or the existing air supply line 12 of a patient care room).
97. The fragrance delivery device 100 according to embodiment 95 or 96, wherein the fragrance delivery device 100 further comprises one or more features as recited in any one of embodiments 1 to 62.

Other Alternative, Less Preferred Fragrance Delivery Devices

98. A fragrance delivery device 100 comprising: a body 101 defining an interior space 112 that houses a fragrance medium 170 and a diffuser 160, the body 101 comprising a nozzle coupler 122 and one or more vents 124, wherein (a) the nozzle coupler 122 is in fluid communication with the diffuser 160, the nozzle coupler 122 being adapted to receive and deliver a first gas flow G1 to the diffuser 160, (b) the diffuser 160 is adapted to disperse the first gas flow G1 received from the nozzle coupler 122 into the interior space 112 of the body 101 to generate a fragrance-carrying second gas flow G2, and (c) the one or more vents 124 are adapted for passage of the fragrance-carrying second gas flow G2 from the interior space 112 to an atmosphere 200 outside the fragrance delivery device 100.
99. The fragrance delivery device 100 according to embodiment 98, wherein the fragrance medium 170 is housed in a frangible container 130″, and the body 101 is adapted to enable fracturing of the frangible container 130″ to thereby release the fragrance medium 170 from the frangible container 130″.
100. The fragrance delivery device 100 according to embodiment 98 or 99, wherein the body 101 comprises an outer casing 110, and the body 101 is adapted to enable fracturing of the frangible container 130 by forming the outer casing 110 at least in part of a material of sufficient flexibility to enable crushing of the frangible container 130″ via application of a compression force to the outer casing 110.
101. The fragrance delivery device 100 according to any one of embodiments 98 to 100, wherein the outer casing 110 is formed at least in part of a material of sufficient flexibility to enable crushing of the frangible container 130″ via application of a compression force to the outer casing 110 in the form of a user manually squeezing the outer casing 110 by hand.
102. The fragrance delivery device 100 according to any one of embodiments 98 to 101, wherein the frangible container 130″ holding the fragrance medium 170 is a first frangible container 130″a, and the fragrance delivery device 100 further comprises a second frangible container 130″b housed in the interior space 112, the second frangible container 130″b housing a suspension medium 172 for mixing with the fragrance medium 170, and the body 101 is adapted to enable fracturing of both the first and second frangible containers 130″a/130″b to thereby release the fragrance and suspension mediums 170/172 from the respective frangible containers 130″a/130″b.
103. The fragrance delivery device 100 according to embodiment 102, wherein the body 101 comprises an outer casing 110, and the body 101 is adapted to enable fracturing of the first and second frangible containers 130″a/130″b by forming the outer casing 110 at least in part of a material of sufficient flexibility to enable crushing of the frangible containers 130″a/130″b via application of a compression force to the outer casing 110.
104. The fragrance delivery device 100 according to embodiment 102 or 103, wherein the outer casing is formed at least in part of a material of sufficient flexibility to enable crushing of the first and second frangible containers 130″a/130″b via application of a compression force to the outer casing in the form of a user manually squeezing the outer casing by hand.
105. The fragrance delivery device 100 according to any one of embodiments 102 to 104, wherein the fragrance delivery device 100 houses the fragrance 170 medium and the suspension medium 172 which are suitably paired for mixing with one another, upon fracturing of the first and second frangible containers 130″a/130″b, to form a fragrance-carrying amalgamate 140.
106. The fragrance delivery device 100 according to embodiment 105, wherein the diffuser 160 is positioned at a lower region of the interior space 112 such that, upon mixing of the fragrance and suspension mediums 170/172, at least a gas-outlet portion 162 of the diffuser 160 is submerged in the fragrance-carrying liquid amalgamate 140.
107. The fragrance delivery device 100 according to any one of embodiments 98 to 106, wherein the nozzle coupler 122 is in fluid communication with the diffuser 160 via a stem 150 that extends along a length of the body 101, and the nozzle coupler 122, stem 150, and diffuser 160 together form a Venturi structure that promotes dispersal of the first gas flow G1 as a compressed gas.
108. The fragrance delivery device 100 according to embodiment 98, wherein the body 101 comprises an outer casing 110 and a canopy 120 at an upper end of the outer casing 110, and the one or more vents 124 are positioned along a periphery of the fragrance delivery device 100 where the canopy 120 meets the outer casing 110.
109. The fragrance delivery device 100 according to embodiment 108, wherein the body 101 comprises a vent 124 in the form of a screen (not shown) that extends around an entire periphery of the body 101, the screen being adapted to support the canopy 120 on the outer casing 110 and to enable passage of the fragrance-carrying second gas flow G2 from the interior space 112 of the body 101 to an atmosphere 200 outside the body 101.

Other Possible Features of Any of the Disclosed Fragrance Delivery Devices

110. The fragrance delivery device 100 according to any one of embodiments 1 to 109, wherein the fragrance delivery device 100 is attachable to the source of compressed gas 20, such as a compressed gas cylinder/container 600 or an existing air supply line 14 of a patient care room. It should be understood that the fragrance delivery device 100 of the present invention may be used with a source of compressed gas 20, such as a compressed gas cylinder/container 600 or existing air supply lines 14 in any environment in which odor is desirably controlled. Environments other than patient care rooms (e.g., hospital rooms, out-patient rooms, veterinarian rooms, surgical settings, etc.) may include, but are not limited to, industrial rooms, manufacturing rooms, home rooms, school rooms, business rooms, sport arena rooms, locker rooms, etc.
111. The fragrance delivery device 100 according to any one of embodiments 1 to 3 and 5 to 110, wherein said fragrance delivery device 100 does not require internal or external electric power to disperse the fragrance-carrying fluid 270 into an atmosphere 200 outside the fragrance delivery device 100.
112. The fragrance delivery device 100 according to any one of embodiments 1 to 111, wherein said fragrance delivery device 100 does not comprise any electronics or any electrically powered components.
113. The fragrance delivery device 100 according to any one of embodiments 1 to 3 and 5 to 110, wherein said fragrance delivery device 100 utilizes an internal or external source of electric power to disperse the fragrance-carrying fluid 270 into an atmosphere 200 outside the fragrance delivery device 100.
114. The fragrance delivery device 100 according to embodiment 113, wherein the timer mechanism 190 utilizes an internal or external source of electric power.
115. The fragrance delivery device 100 according to embodiment 113 or 115, wherein the internal or external source of electric power comprises one or more batteries (not shown).
116. The fragrance delivery device 100 according to any one of embodiments 1 to 115, wherein the fragrance-carrying fluid 270 comprises one or more of: (i) at least one alcohol, (ii) at least one odor molecule sequestering agent, (iii) at least one odor neutralizing agent, and (iv) at least one solvent.
117. The fragrance delivery device 100 according to any one of embodiments 1 to 116, wherein the fragrance-carrying fluid 270 comprises: (i) at least one alcohol, (ii) at least one odor molecule sequestering agent, (iii) at least one odor neutralizing agent, and (iv) at least one solvent.
118. The fragrance delivery device 100 according to embodiment 116 or 117, wherein the at least one alcohol comprises isopropanol or ethanol.
119. The fragrance delivery device 100 according to any one of embodiments 116 to 118, wherein the at least one alcohol comprises isopropanol.
120. The fragrance delivery device 100 according to any one of embodiments 116 to 119, wherein the at least one odor molecule sequestering agent comprises hydroxypropyl-beta-cyclodextrin, methyl-beta-cyclodextrin, other cyclodextrins and cyclodextrin derivatives, alpha-cyclodextrins, gamma-cyclodextrins, zeolites, activated carbon, montmorillonite clay, Fuller's earth, bentonite clay, and any combinations thereof.
121. The fragrance delivery device 100 according to any one of embodiments 116 to 120, wherein the at least one odor molecule sequestering agent comprises hydroxypropyl-beta-cyclodextrin.
122. The fragrance delivery device 100 according to any one of embodiments 116 to 121, wherein the at least one odor molecule sequestering agent comprises methyl-beta-cyclodextrin.
123. The fragrance delivery device 100 according to any one of embodiments 116 to 122, wherein the at least one odor neutralizing agent comprises sodium bicarbonate, potassium bicarbonate, calcium hydroxide, sodium perborate, sodium percarbonate, hydrogen peroxide, calcium peroxide, and any combination thereof.
124. The fragrance delivery device 100 according to any one of embodiments 116 to 123, wherein the at least one odor neutralizing agent comprises sodium bicarbonate.
125. The fragrance delivery device 100 according to any one of embodiments 116 to 124, wherein the at least one odor neutralizing agent comprises sodium perborate.
126. The fragrance delivery device 100 according to any one of embodiments 1 to 125, further comprising an adapter 144 for attaching a nozzle 14 of the gas supply unit 10 to the fragrance delivery device 100 (e.g., via a nozzle coupler 122 of the fragrance delivery device 100). See, for example, FIG. 17.
127. The fragrance delivery device 100 according to any one of embodiments 1 to 126, wherein the source of compressed gas 20 is the existing air supply line 12 of a room (e.g., a patient care room).
128. The fragrance delivery device 100 according to any one of embodiments 1 to 127, wherein the fragrance delivery device 100 is connected to the existing air supply line 12 of a room (e.g., a patient care room).
129. The fragrance delivery device 100 according to any one of embodiments 1 to 126, wherein the source of compressed gas 20 is the compressed gas container 600.
130. The fragrance delivery device 100 according to embodiment 129, wherein the compressed gas container 600 comprises a gas tank/cylinder 600. See, for example, FIG. 18.
131. The fragrance delivery device 100 according to embodiment 129, wherein the compressed gas container 600 comprises an aerosol can (not shown).
132. The fragrance delivery device 100 according to any one of embodiments 129 to 131, wherein the fragrance delivery device 100 is connected to the compressed gas container 600.

Methods of Using Fragrance Delivery Devices

133. A method of dispensing a fragrance comprising: utilizing the fragrance delivery device 100 of any one of embodiments 1 to 132 to emit a fragrance-carrying fluid 270 into an atmosphere 200 outside the fragrance delivery device 100.
134. The method according to embodiment 133, wherein the fragrance delivery device 100 is utilized while in fluid communication with a compressed gas source 20.
135. The method according to embodiment 134, wherein the compressed gas source 20 comprises the existing air supply line 12 of a room (e.g., a patient care room).
136. The method according to embodiment 134, wherein the compressed gas source 20 comprises a gas tank/cylinder 600.
137. The method according to embodiment 134, wherein the compressed gas source 20 comprises an aerosol can (not shown).
138. The method according to any one of embodiments 133 to 135, wherein the fragrance delivery device 100 is utilized while in fluid communication with the compressed gas source 20 via a gas supply unit 10, the gas supply unit 10 receiving a compressed gas flow from the compressed gas source 20 via a gas line 12 and delivering the compressed gas flow to the fragrance delivery device 100 via a mating connection (e.g., adapter 144) between a nozzle 14 of the gas supply unit 10 and the nozzle coupler 122 of the fragrance delivery device 100.
139. A method of dispensing a fragrance, said method comprising: utilizing the fragrance delivery device 100 of any one of embodiments 1 to 94 and 110 to 132 to emit a fragrance-carrying fluid 270 into an atmosphere 200 outside the fragrance delivery device 100.
140. The method according to any one of embodiments 133 to 139, wherein said utilizing step comprises: dispersing the fragrance-carrying fluid 270 into the atmosphere 200 outside the fragrance delivery device 100 via a removable cartridge 130′.
141. The method according to any one of embodiments 133 to 139, wherein said utilizing step comprises: fracturing the first and second frangible containers 130″a/130″b to release and mix the fragrance and suspension mediums 170/172 to form a fragrance-carrying liquid amalgamate 140 that submerges at least a gas-outlet portion 162 of the diffuser 160, feeding a first gas flow G1 into the nozzle coupler 122 for delivery to the diffuser 160, and discharging the first gas flow G1 into the liquid amalgamate 140 to form a fragrance-carrying second gas flow G2, and emitting the fragrance-carrying second gas flow G2 from the fragrance delivery device 100 via the one or more vents 124.

Methods of Making Fragrance Delivery Devices

142. A method of making the fragrance delivery device 100 of any one of embodiments 1 to 132, said method comprising: forming one or more of the components recited in any one of embodiments 1 to 132.
143. The method of embodiment 142, wherein said forming step comprises one or more of: a thermoforming (e.g., molding) step, a machining step, a bonding step, an assembling step, or any combination thereof.
144. The method of embodiment 142 or 143, wherein said forming step comprises: assembling the assembly of mechanical components 700, the assembly of mechanical components 700 comprising one or more of: (a) a timing mechanism 190 that runs for a desired length of time (e.g., from 2.0 seconds to 60 minutes, typically about a 30 minute duration while providing approximately 210 degrees of output shaft 193 rotation thru this 30 minute cycle); (b) an output shaft 193 extending from the timing mechanism 190, and rotating for a desired length of time (e.g., from 2.0 seconds to 60 minutes)(see, FIGS. 9-10); (c) a driver shaft 194 that is axially connected to the output shaft 193 (see, FIGS. 9-10); (d) a cam shaft 195, which is preferably in an “offset” position relative to the driver shaft 194 (see, FIGS. 9-10); (e) a combination of spur gears 196 that connect the driver shaft 194 to the cam shaft 195 (e.g., the cam shaft 195 is “disconnected” from the driver shaft 194 when the combination of spur gears 196 are separated from one another, which allows a coil spring assembly 197 to rotate the cam shaft 195 back to a “reset/start” position; when the driver shaft 194 is “connected” to the cam shaft 195, the cam shaft 195 also rotates for a desired length of time (e.g., from 2.0 seconds to 60 seconds))(see, FIGS. 9-10); (f) one or more cam gears 191 designed to (i) rotate when the cam shaft 195 rotates, and (i) open and close one or more valves 135 during rotation (see, FIGS. 9-10); (g) the one or more valves 135 that control when pneumatic pressure is applied to disperse a fragrance-carrying fluid 270 (not shown in FIG. 8)(see, FIGS. 9-10) into the atmosphere 200 outside the fragrance delivery device 100; (h) an air supply valve 134 that controls air flow of a first gas flow G1 from a source of compressed gas 20, such as a compressed gas cylinder/container 600 or an existing air supply line 12; (i) a coil spring assembly 197 designed to rotate the cam shaft 195 back to a “reset/start” position following a dispersion step, the coil spring assembly 197 comprising (h)(i) a cam shaft reset wheel 197a, and (h)(ii) a coiled spring 197b; (j) a driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198 (see, FIGS. 9-10); (k) a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows the pinon gear 199b to spin in an opposite unwind direction (see, FIGS. 9-10); (l) a lever 630 connected to a toothed gear 199a, which couples with a second gear 199b that drives shaft 194, which winds a spring 197b within the timer 190 and through a one-way clutch 640, which does not turn a spur gear 196 while the timer 190 is being wound, but does allow rotation of spur gear 196 when the timer 190 is unwinding; (m) an adjustable stop pin 201 that provides a correct rotation start position for the cam shaft 195 (see, FIG. 9); and (n) one or more pressure intensifiers 500, each pressure intensifier 500 being capable of generating an intensified (e.g., an increased) pressure used to disperse fragrance-carrying fluid 270 into an atmosphere 200 outside the fragrance delivery device 100. 145. The method of any one of embodiments 142 to 144, wherein said forming step comprises: incorporating one or more of the following into the fragrance delivery device 100: (a) a timing mechanism 190 that runs for a desired length of time (e.g., from 2.0 seconds to 60 minutes, typically about a 30 minute duration while providing approximately 210 degrees of output shaft 193 rotation thru this 30 minute cycle); (b) an output shaft 193 extending from the timing mechanism 190, and rotating for a desired length of time (e.g., from 2.0 seconds to 60 minutes)(see, FIGS. 9-10); (c) a driver shaft 194 that is axially connected to the output shaft 193 (see, FIGS. 9-10); (d) a cam shaft 195, which is preferably in an “offset” position relative to the driver shaft 194 (see, FIGS. 9-10); (e) a combination of spur gears 196 that connect the driver shaft 194 to the cam shaft 195 (e.g., the cam shaft 195 is “disconnected” from the driver shaft 194 when the combination of spur gears 196 are separated from one another, which allows a coil spring assembly 197 to rotate the cam shaft 195 back to a “reset/start” position; when the driver shaft 194 is “connected” to the cam shaft 195, the cam shaft 195 also rotates for a desired length of time (e.g., from 2.0 seconds to 60 seconds))(see, FIGS. 9-10); (f) one or more cam gears 191 designed to (i) rotate when the cam shaft 195 rotates, and (i) open and close one or more valves 135 during rotation (see, FIGS. 9-10); (g) the one or more valves 135 that control when pressure is applied to disperse a fragrance-carrying fluid 270 (not shown in FIG. 8)(see, FIGS. 9-10) into the atmosphere 200 outside the fragrance delivery device 100; (h) an air supply valve 134 that controls air flow of a first gas flow G1 from a source of compressed gas 20, such as a compressed gas cylinder 600 or an existing air supply line 12; (i) a coil spring assembly 197 designed to rotate the cam shaft 195 back to a “reset/start” position following a dispersion step, the coil spring assembly 197 comprising (h)(i) a cam shaft reset wheel 197a, and (h)(ii) a coiled spring 197b; (j) a driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198 (see, FIGS. 9-10); (k) a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows the pinon gear 199b to spin in an opposite unwind direction (see, FIGS. 9-10); (1) a lever 630 connected to a toothed gear 199a, which couples with a second gear 199b that drives shaft 194, which winds a spring 197b within the timer 190 and through a one-way clutch 640, which does not turn a spur gear 196 while the timer 190 is being wound, but does allow rotation of spur gear 196 when the timer 190 is unwinding; (m) an adjustable stop pin 201 that provides a correct rotation start position for the cam shaft 195 (see, FIG. 9); and (n) one or more pressure intensifiers 500, each pressure intensifier 500 being capable of generating an intensified (e.g., an increased) pressure used to disperse fragrance-carrying fluid 270 into an atmosphere 200 outside the fragrance delivery device 100.
146. The method of any one of embodiments 142 to 145, wherein said forming step comprises: incorporating one or more pressure intensifiers 500 into the fragrance delivery device 100, each pressure intensifier 500 being capable of generating an intensified (e.g., an increased) pressure used to disperse the fragrance-carrying fluid 270 into the atmosphere 200 outside the fragrance delivery device 100.
147. The method of any one of embodiments 142 to 146, wherein said forming step comprises: forming a removable cartridge port 180 within an outer surface of the outer casing 110 of the fragrance delivery device 100, the removable cartridge port 180 being sized to accept a removable cartridge 130′ containing a fragrance medium 170 therein.
148. The method of any one of embodiments 142 to 147, wherein said forming step comprises: forming a removable cartridge 130′ containing a fragrance medium 170 therein. See, for example, exemplary removable cartridge 130′ shown in FIGS. 14A-14B.

Removable Cartridges For Use in Fragrance Delivery Devices

149. A removable cartridge 130′, the removable cartridge 130′ comprising the removable cartridge 130′ described in any one of embodiments 63 to 90 and 93 to 94.

Piston-Sealing Feature Suitable for Use in Fragrance Delivery Devices as Well as Other Devices

150. The piston-sealing feature described in embodiments 47 to 51.
151. A piston (see, for example, exemplary pressure intensifier piston 506) comprising: one or more o-ring grooves (see, for example, exemplary o-ring grooves 554) that circumscribe an outer side surface (see, for example, exemplary outer side surface 555) of the piston positioned within a master cylinder volume of a master cylinder (not shown), each of the one or more o-ring grooves being sized to contain one or more o-rings. (See, for example, exemplary pressure intensifier piston 506 shown in FIGS. 13A-13C.)
152. The piston according to embodiment 151, wherein each of the one or more o-ring grooves is designed to (1) apply a sealing force onto one or more o-rings positioned within a given o-ring groove, the sealing force forcing the one or more o-rings between (i) an inner surface of the master cylinder volume and (ii) an outer side surface of the piston while the piston is pushed by pneumatic (or hydraulic) pressure of a gas (or liquid), and (2) apply a minimal force, if any, onto the one or more o-rings positioned within a given o-ring groove and between (i) an inner surface of the master cylinder volume and (ii) an outer side surface of the piston while the piston retreats back into an initial position once the pneumatic (or hydraulic) pressure of the gas (or liquid) is stopped. See again, for example, FIGS. 13A-13C.
153. The piston according to embodiment 151 or 152, wherein the one or more o-ring grooves comprises two or more o-ring grooves that each circumscribe an outer side surface of the piston.
154. The piston according to any one of embodiments 151 to 153, further comprising one or more o-rings positioned within each of the one or more o-ring grooves.
155. The piston according to any one of embodiments 151 to 154, further comprising two or more o-rings positioned within each of the one or more o-ring grooves.
156. The piston according to any one of embodiments 151 to 155, wherein each of the one or more o-ring grooves has a groove configuration such that a first distance between (i) an inner surface of the master cylinder volume and (ii) an outer side surface of the piston at one location within a given o-ring groove (i.e., proximate a forward end of the given o-ring groove) is greater than a second distance between (i) the inner surface of the master cylinder volume and (ii) the outer side surface of the piston at another location within a given o-ring groove (i.e., proximate a rear end of the given o-ring groove). See again, the exemplary groove configurations shown in FIGS. 13A-13C.
157. The piston according to any one of embodiments 151 to 156, in combination with the fragrance delivery device 100 of the present invention, a hand pneumatic air pump (not shown), a liquid pump (not shown), a hand operated pump on a well (not shown), an automobile air conditioner compressor, or any combination thereof.

It should be understood that although the above-described fragrance delivery devices 100, and methods are described as “comprising” one or more features, components or steps, the above-described fragrance delivery devices 100, and methods may “comprise,” “consists of,” or “consist essentially of” any of the above-described features, components or steps of the fragrance delivery devices 100, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a fragrance delivery device 100 and/or method that “comprises” a list of elements (e.g., components or steps) is not necessarily limited to only those elements (or components or steps), but may include other elements (or components or steps) not expressly listed or inherent to the fragrance delivery device 100 and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fragrance delivery device 100 and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of.”

Further, it should be understood that the herein-described fragrance delivery devices 100, and/or methods may comprise, consist essentially of, or consist of any of the herein-described components, features, and steps, as shown in the figures with or without any feature(s) not shown in the figures. In other words, in some embodiments, the fragrance delivery devices 100 and/or methods of the present invention do not have any additional features other than those shown in the figures, and such additional features, not shown in the figures, are specifically excluded from the fragrance delivery devices 100 and/or methods. In other embodiments, the fragrance delivery devices 100 and/or methods of the present invention do have one or more additional features that are not shown in the figures.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1: Preparation of a Deodorizer Formulation and Use of the Deodorizer Formulation in a Fragrance Delivery Device

The following components shown in Table 1 were combined to form a fragrance and suspension medium.

TABLE 1
Fragrance and Suspension Medium Components

| | Weight Percent |
|---|---|
| Ingredient | (Wt %) |
| 2-hydroxypropyl-beta-cyclodextrin | 10 |
| Methyl-beta-cyclodextrin | 10 |
| 91% Isopropyl alcohol (91% IPA + 9% | 15 |
| water) | |
| Sodium bicarbonate | 0.5 |
| Sodium perborate | 0.75 |
| Water | 63.75 |

Water was used as the principal solvent. Alternative solvents and/or cosolvents could include, but are not limited to, inhalable propellants such as fluorocarbons and freons.

Isopropanol served four functions in this system: (a) malodor cosolvent—removing some of the odor molecules from the air, (b) preservative to prevent microbial growth, (c) surface tension reducing agent—assists with atomization, and (d) provides it's own distinctive smell, roughly associated with medicine, getting injections, and cleanliness in general. Alternatives to isopropanol include, but are not limited to, ethanol.

Hydroxypropyl-beta-cyclodextrin was used as an odor molecule sequestering agent. Alternatives include, but are not limited to, other cyclodextrins and cyclodextrin derivatives, alpha-cyclodextrins, gamma-cyclodextrins, and any combinations thereof. Still further alternatives include, but are not limited to, zeolites, activated carbon, montmorillonite clay, Fuller's earth, bentonite clay, and any combinations thereof.

Methyl-beta-cyclodextrin was used as a high solubility odor molecule sequestering agent.

Sodium bicarbonate was used as a mild base to help neutralize acidic odor molecules, as well as certain very highly basic odor molecules such as ammonia. Alternatives include, but are not limited to, potassium bicarbonate, calcium hydroxide, and any combination thereof.

Sodium perborate was used as a mild oxidizing agent to help neutralize reducing odors, such as hydrogen sulfide, mercaptans, and thiols. Alternatives include, but are not limited to, sodium percarbonate, hydrogen peroxide, calcium peroxide, and any combination thereof.

A fragrance delivery device, similar to fragrance delivery device 100 discussed above, was used to disperse the composition in Table 1 within a patient care room (e.g., a patient clinic, a patient operating room, etc.) via an existing air supply unit 10 or a compressed gas cylinder/container 600 within the patient care room. Compressed hospital air was supplied to the fragrance delivery device at an air pressure of about 55 psi.

### Example 2: Preparation of a Deodorizer Formulation and Use of the Deodorizer Formulation in a Fragrance Delivery Device

The following components shown in Table 2 were combined to form a fragrance and suspension medium.

TABLE 2
Fragrance and Suspension Medium Components

| | Weight Percent |
|---|---|
| Ingredient | (Wt %) |
| 2-hydroxypropyl-beta-cyclodextrin | 10 |
| 95% Ethyl alcohol (95% ethanol + 5% | 15 |
| water) | |
| Sodium bicarbonate | 0.5 |
| Sodium perborate | 0.5 |
| Water | 73.5 |

A fragrance delivery device, similar to fragrance delivery device 100 discussed above, was used to disperse the composition in Table 2 within a veterinary patient care room via an existing air supply unit 10 within the veterinary patient care room. Compressed air was supplied to the fragrance delivery device at an air pressure of about 55 psi.

What is claimed is:

1. A fragrance delivery device 100 comprising:
a body 101 defining an interior space 112, the body 101 comprising a nozzle coupler 122 designed to be attachable to a source of compressed gas 20, the source of compressed gas 20 comprising an existing air supply line 14 of a room, or a compressed gas container 600, the fragrance delivery device 100 being adapted to utilize pneumatic pressure P_{O} of a first gas flow G1 from the source of compressed gas 20, to disperse and/or dispense a fragrance-carrying fluid 270 into an atmosphere 200 outside the fragrance delivery device 100.

2. The fragrance delivery device 100 according to claim 1, wherein the pneumatic pressure P_{O} of the source of compressed gas 20 is used to (1) generate an intensified pressure P_{I}, and (2) utilize the intensified pressure P_{I} to disperse a fragrance-carrying fluid 270 into the atmosphere 200 outside the fragrance delivery device 100.

3. The fragrance delivery device 100 according claim 2, wherein the fragrance delivery device 100 disperses the fragrance-carrying fluid 270 into the atmosphere 200 outside the fragrance delivery device 100 without using any internal or external electric power to disperse the fragrance-carrying fluid 270.

4. The fragrance delivery device 100 according to claim 1, wherein the fragrance delivery device 100 comprises an assembly of mechanical components 700, said assembly of mechanical components 700 comprises one or more of:
(a) a timing mechanism 190 that runs for a desired length of time;
(b) an output shaft 193 extending from the timing mechanism 190, and rotating for a desired length of time;
(c) a driver shaft 194 that is axially connected to the output shaft 193;
(d) a cam shaft 195, which is preferably in an “offset” position relative to the driver shaft 194;
(e) a combination of spur gears 196 that connect the driver shaft 194 to the cam shaft 195;
(f) one or more cam gears 191 designed to (i) rotate when the cam shaft 195 rotates, and (i) open and close one or more valves 135 during rotation;
(g) the one or more valves 135 that control when pneumatic pressure is applied to disperse a fragrance-carrying fluid 270 into the atmosphere 200 outside the fragrance delivery device 100;
(h) an air supply valve 134 that controls air flow of a first gas flow G1 from the source of compressed gas 20;
(i) a coil spring assembly 197 designed to rotate the cam shaft 195 back to a “reset/start” position following a dispersion step, the coil spring assembly 197 comprising (h)(i) a cam shaft reset wheel 197a, and (h)(ii) a coiled spring 197b;
(j) a driver shaft clutch 198 that provides one-way torque in a direction of rotation of the output shaft 193 and the driver shaft 194, and zero torque in an opposite direction, allowing the driver shaft 194 to freely spin when not being rotated via driver shaft clutch 198;
(k) a rack-n-pinion rewind assembly 199 used to rewind the output shaft 193 extending out of the timing mechanism 190, the rack-n-pinion rewind assembly 199 providing torque in a timer wind direction via a one-way timer clutch 199a, and allows the pinon gear 199b to spin in an opposite unwind direction along gear strip 199C;
(l) a lever 630 connected to a toothed gear 199a, which couples with a second gear 199b that drives shaft 194, which winds a spring 197b within the timer 190 and through a one-way clutch 640, which does not turn a spur gear 196 while the timer 190 is being wound, but does allow rotation of spur gear 196 when the timer 190 is unwinding;
(m) an adjustable stop pin 201 that provides a correct rotation start position for the cam shaft 195; and
(n) one or more pressure intensifiers 500, each pressure intensifier 500 being capable of generating an intensified pressure used to disperse fragrance-carrying fluid 270 into an atmosphere 200 outside the fragrance delivery device 100.

5. The fragrance delivery device 100 according to claim 4, wherein the fragrance delivery device 100 comprises a timing mechanism 190 that runs for a desired length of time ranging from about 2.0 seconds (sec) to about 60 minutes (min).

6. The fragrance delivery device 100 according to claim 4, wherein the components within the exemplary assembly of mechanical components 700 are either (1) connected to a fixed vertical base plate 202, attached to a fixed horizontal base plate 204, or (2) positioned on and connected to a pivoting plate 203, the pivoting plate 203 being connected, via a hinge 205, to the fixed vertical base plate 202, and
wherein a position of the pivoting plate 203 is controlled (1) with an activation lever 192, or (2) by the presence or absence of a removable cartridge 130′ positioned within a removable cartridge port 180 of the fragrance delivery device 100.

7. The fragrance delivery device 100 according to claim 6, wherein when the pivoting plate 203 is in a horizontal position, the exemplary assembly of mechanical components 700 is “active” and (1) a main pneumatic pressure P_{O} from the source of compressed gas 20 is on, and (2) the combination of spur gears 196 that connect the driver shaft 194 to the cam shaft 195 are engaged, and when the pivoting plate 203 is pivoted into a downward position, two things happen simultaneously, namely: (1) a main pneumatic pressure P_{O} from the source of compressed gas 20 is off, and (2) the combination of spur gears 196 that connect the driver shaft 194 to the cam shaft 195 is disengaged.

8. The fragrance delivery device 100 according to claim 1, wherein the body 101 further comprises a removable cartridge port 180 sized to accept a removable cartridge 130′ containing a fragrance medium 170, and the pneumatic pressure P_{O} of the first gas flow G1 is used to disperse the fragrance-carrying fluid 270 from a removable cartridge 130′ positioned within the removable cartridge port 180, the removable cartridge 130′ comprising one or more cartridge nozzles 124′ sized to allow passage of the fragrance-carrying fluid 270 from the removable cartridge 130′ into the atmosphere 200 outside the fragrance delivery device 100.

9. The fragrance delivery device 100 according to claim 1, wherein the source of compressed gas 20 comprises the compressed gas container 600.

10. The fragrance delivery device 100 according to claim 1, wherein the source of compressed gas 20 comprises the existing air supply line 14 of a room,

11. A fragrance delivery device 100 comprising:
a body 101 defining an interior space 112, the body 101 comprising
(i) a nozzle coupler 122 designed to be attachable to a source of compressed gas 20, the source of compressed gas 20 comprising a compressed gas cylinder/container 600 or an existing air supply line 12 of a patient care room, and
(ii) a removable cartridge port 180 sized to accept a removable cartridge 130′ containing a fragrance medium 170,
the nozzle coupler 122 being adapted to receive a first gas flow G1 from the source of compressed gas 20 and utilize a pneumatic pressure P_{O} of the first gas flow G1 to disperse a fragrance-carrying fluid 270 from a removable cartridge 130′ positioned within the removable cartridge port 180, the removable cartridge 130′ comprising one or more cartridge nozzles 124′ sized to allow passage of the fragrance-carrying fluid 270 from the removable cartridge 130′ into an atmosphere 200 outside the fragrance delivery device 100.

12. The fragrance delivery device 100 according to claim 11, wherein insertion of the removable cartridge 130′ into the removable cartridge port 180 initiates one or more of: (i) opens an air supply valve 134 from the source of compressed gas 20 to one or more valves 135 leading to one or more of the removable cartridges 130′, (ii) engages a timing mechanism 190 that runs for a desired length of time, and (iii) engages two or more gears with one another.

13. The fragrance delivery device 100 according to claim 11, wherein the source of compressed gas 20 comprises the compressed gas container 600.

14. The fragrance delivery device 100 according to claim 11, wherein the source of compressed gas 20 comprises the existing air supply line 14 of a room,

15. A fragrance delivery device 100 comprising:
a body 101 defining an interior space 112, the body 101 comprising a nozzle coupler 122 designed to be attachable to a source of compressed gas 20, the source of compressed gas 20 comprising a compressed gas cylinder or an existing air supply line 12 of a patient care room, the fragrance delivery device 100 being adapted to (1) utilize pneumatic pressure P_{O} from the source of compressed gas 20 to generate an intensified (e.g., an increased) pressure P_{I}, and (2) utilize the intensified pressure P_{I} to disperse a fragrance-carrying fluid 270 into an atmosphere 200 outside the fragrance delivery device 100.

16. The fragrance delivery device 100 according to claim 15, wherein the intensified pressure P_{I} is used to push on a cartridge piston 411 or a cartridge ball 431 of a removable cartridge 130′ so as to force fragrance-carrying fluid 270 from one or more cartridge nozzles 124′ of the removable cartridge 130′ into an atmosphere 200 outside the fragrance delivery device 100.

17. The fragrance delivery device 100 according to claim 15, wherein the source of compressed gas 20 comprises the compressed gas container 600.

18. The fragrance delivery device 100 according to claim 15, wherein the source of compressed gas 20 comprises the existing air supply line 14 of a room,

19. A method of dispensing a fragrance comprising:
utilizing the fragrance delivery device 100 of claim 11 to emit a fragrance-carrying fluid 270 into an atmosphere 200 outside the fragrance delivery device 100.

20. A method of dispensing a fragrance comprising:
utilizing the fragrance delivery device 100 of claim 15 to emit a fragrance-carrying fluid 270 into an atmosphere 200 outside the fragrance delivery device 100.
